(19) United States
(12) Patent Application Publication
(10) Pub. No.: US 2004/0018530 A1
(43) Pub. Date: Jan. 29, 2004

(54) In vitro evolution of functional RNA and DNA using electrophoretic selection

(76) Inventors: Michael T. Bowser, Minneapolis, MN (US); Shaun D. Mendonsa, St. Louis Park, MN (US)

Correspondence Address: MUETING, RAASCH & GEBHARDT, P.A., P.O. BOX 581415, MINNEAPOLIS, MN, 55458, US

(21) Appl. No.: 10/448,250

(22) Filed: Sep. 29, 2003

Related U.S. Application Data
(60) Provisional application No. 60/470,750, filed on May 15, 2003.
(60) Provisional application No. 60/384,709, filed on May 31, 2002.

(51) Int. Cl. C12Q 1/70; C12Q 1/68; C12P 19/34

(52) U.S. Cl. 435/6; 435/5; 435/91.2

(57) ABSTRACT

The CE-SELEX procedure of the present invention is a novel selection procedure that utilizes capillary electrophoresis (CE) in combination with conventional SELEX selection procedures. CE-SELEX, for the first time, allows selection to be performed in free solution. Performing selection against a target in free solution yields aptamers with improved affinity and/or improved selectivity toward target molecules for use as improved pharmaceuticals and diagnostic agents.

This application claims the benefit of the U.S. Provisional Application Serial No. 60/384,709, filed May 31, 2002, and U.S. Provisional Application Serial No. 60/470,750, filed May 15, 2003, both of which are incorporated herein by reference in their entirety.

### STATEMENT OF GOVERNMENT RIGHTS

The present invention was made with support from National Institutes of Health, Grant No. R01 GM63533-01A1. The government may have certain rights in this invention.

### BACKGROUND

One of the most challenging research areas facing chemists today is the screening of large combinatorial libraries for molecules with a given functionality. The premise of combinatorial screening is that in a sufficiently large initial set of compounds there should be one or more species with the desired properties. This approach leads to the labor intensive process of selecting an initial set of related compounds, synthesizing enough of each compound for analysis and finally assessing which compounds possess the desired functionality. Although there have been significant advances in this area, the large number of permutations in even simple systems make screening combinatorial libraries a labor intensive task. For example, a library consisting of every possible twenty-five amino acid peptide contains 3.4×10³² (20²⁵) molecules. Based on the average molecular mass of an amino acid, a library containing a single molecule of each possible permutation would have a mass of 1.9 million metric tons, over five times the mass of the empire state building.

In view of these challenges, there is a need for improved procedures for the screening of large combinatorial libraries for molecules with a given functionality.

### SUMMARY OF THE INVENTION

The current invention presents the CE-SELEX method, as an improvement over conventional SELEX, for the selection of nucleic acid ligands (also called aptamers) that bind to a specific target. CE-SELEX is based on an electrophoretic separation and, for the first time, allows selection to take place in free solution. Performing electrophoretic selection in free solution eliminates many of the evolutionary biases introduced by the chromatographic separation in conventional SELEX and yields aptamers with improved binding efficiency and selectivity. Performing the electrophoretic selection in free solution also significantly improves the speed of the selection, yielding aptamers in as few as two rounds of selection.

Accordingly, the present invention provides a method for identifying nucleic acid ligands of a target molecule from a candidate mixture of single stranded nucleic acids each having a region of randomized sequence, the method including: contacting the candidate mixture of single stranded nucleic acids each having a region of randomized sequence with the target molecule, wherein nucleic acids having an increased affinity to the target molecule relative to the candidate mixture may be partitioned from the remainder of the candidate mixture; partitioning the increased affinity nucleic acids from the remainder of the candidate mixture by capillary electrophoresis; and amplifying the increased affinity nucleic acids, in vitro, to yield a ligand-enriched mixture of nucleic acids, whereby nucleic acid ligands of the target molecule may be identified.

In another aspect, the present invention also provides a method for identifying nucleic acid ligands of a target molecule from a candidate mixture of single stranded nucleic acids each having a region of randomized sequence, the method including: contacting the candidate mixture of single stranded nucleic acids each having a region of randomized sequence with the target molecule, wherein nucleic acids having an increased affinity to the target molecule relative to the candidate mixture may be partitioned from the remainder of the candidate mixture; partitioning the increased affinity nucleic acids from the remainder of the candidate mixture by capillary electrophoresis; amplifying the increased affinity nucleic acids, in vitro, to yield a ligand-enriched mixture of nucleic acids; and identifying a nucleic acid ligand of the target molecule from the ligand-enriched mixture of nucleic acids.

In some embodiments of the methods of the present invention, the target molecule may be a large molecule, including for example, IgE, Lrp, E. coli metJ protein, elastase, human immunodeficiency virus reverse transcriptase (HIV-RT), human cytomegalovirus (HCMV), and thrombin. In some embodiments, the target molecule may be a small molecule, including, for example, ATP, L-arginine, kanamycin, lividomycin, neomycin, nicotinamide (NAD), N-methylmesoporphyrin (NMM), theophylline, tobramycin, D-tryptophan, L-valine, vitamin B12, D-serine, L-serine, and γ-aminobutyric acid (γ-ABA). In some embodiments, the target molecule may be a neurotransmitter or a neuropeptide. In some embodiments, the target molecule may be a virus, a bacterium, a eukaryotic cell, an organelle, or a nanoparticle.

In some embodiments of the methods of the present invention, the steps of contacting the candidate mixture of single stranded nucleic acids each having a region of randomized sequence with the target molecule, partitioning the increased affinity nucleic acids from the remainder of the candidate mixture by capillary electrophoresis, and amplifying the increased affinity nucleic acids may be repeated. In some embodiments, the steps of contacting the candidate mixture of single stranded nucleic acids each having a region of randomized sequence with the target molecule, partitioning the increased affinity nucleic acids from the remainder of the candidate mixture by capillary electrophoresis, and amplifying the increased affinity nucleic acids may be repeated 2-20 times.

In some embodiments of the methods of the present invention, the single-stranded nucleic acids may be deoxyribonucleic acids, including modified deoxyribonucleic acids, or may be ribonucleic acids, including modified ribonucleic acids.

In some embodiments of the methods of the present invention, the amplifying the nucleic acids with increased affinity may be by polymerase chain reaction (PCR). In some embodiments, the polymerase chain reaction may be performed with primers with a melting temperature of greater than 52° C. and the PCR annealing reaction may be carried out at a temperature of 52° C. or greater. In some embodiments, the primers may have a melting temperature of about 59° C. and the PCR annealing reaction may be carried out at a temperature of about 52° C. to about 54° C.

In some embodiments of the methods of the present invention, the partitioning the increased affinity nucleic acids from the remainder of the candidate mixture by capillary electrophoresis may be performed in a microfluidic device or chip.

In some embodiments of the methods of the present invention, the partitioning the increased affinity nucleic acids from the remainder of the candidate mixture by capillary electrophoresis may be carried out in a CE buffer of about 0 mM to about 40 mM NaCl, including, for example, a CE buffer that is about 30 mM NaCl.

In another aspect, the present invention also provides nucleic acid ligands isolated by the methods of the present invention. In some embodiments, these nucleic acid ligands may be DNA oligonucleotides, RNA oligonucleotides, and modifications thereof. In some embodiments, these nucleic acid ligands may be ligands that bind to a large molecule target molecule, a small molecule target molecule or a macromolecular target. In some embodiments, the nucleic acid ligands may have a binding affinity for IgE, Lrp, E. coli metJ protein, elastase, human immunodeficiency virus reverse transcriptase (HIV-RT), human cytomegalovirus (HCMV), thrombin, ATP, L-arginine, kanamycin, lividomycin, neomycin, nicotinamide (NAD), N-methylmesoporphyrin (NMM), theophylline, tobramycin, D-tryptophan, L-valine, vitamin B12, D-serine, L-serine, γ-aminobutyric acid (γ-ABA), a virus, a bacteria, a eukaryotic cell, an organelle, or a nanoparticle.

In another aspect, the present invention also provides nucleic acid ligands having SEQ ID NO:1-117, and modifications thereof.

In yet another aspect, the present invention provides CE-SELEX kits with one or more components selected from capillary tubes suitable for CE, a primer pair, a DNA combinatorial library, an RNA combinatorial library, PCR reagents, CE separation buffer, streptavidin/agarose columns, transcriptase, and a reverse transcriptase. In some embodiments, the CE-SELEX kit may include instructions for use. In some embodiments, these instructions for use may include instructions for the modification of the instrument control software or the data collection software of a CE instrument to facilitate fraction collection in the CE-SELEX procedure.

### DETAILED DESCRIPTION OF THE DRAWINGS

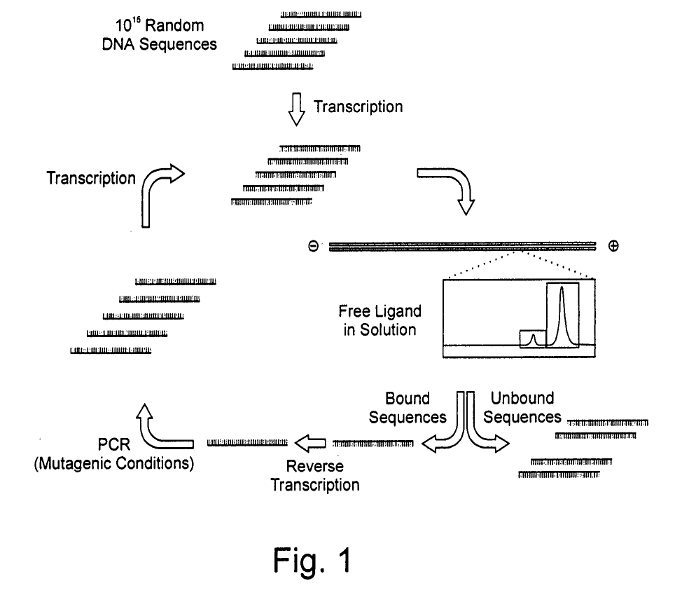

FIG. 1. Schematic representation of the methodology of CE-SELEX. CE-SELEX may be used to select DNA aptamers as well as RNA aptamers. To select a DNA aptamer the reverse transcription and transcription steps are omitted from the procedure depicted in the figure.

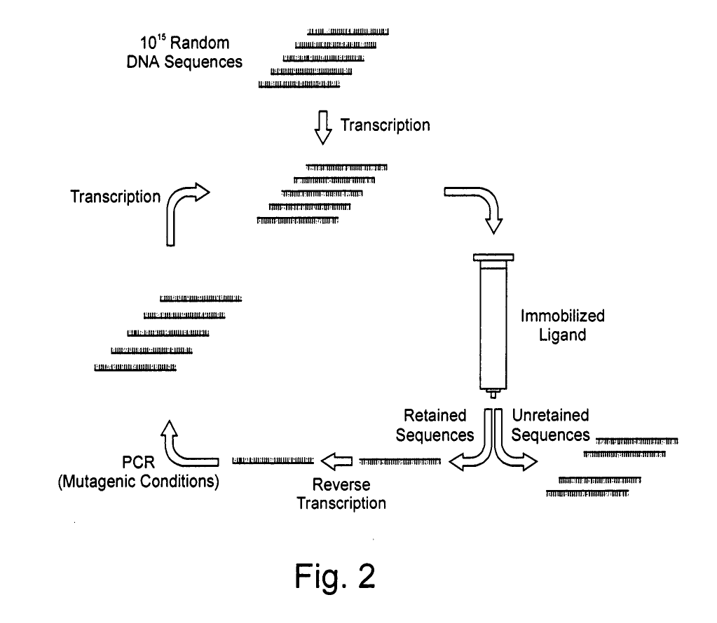

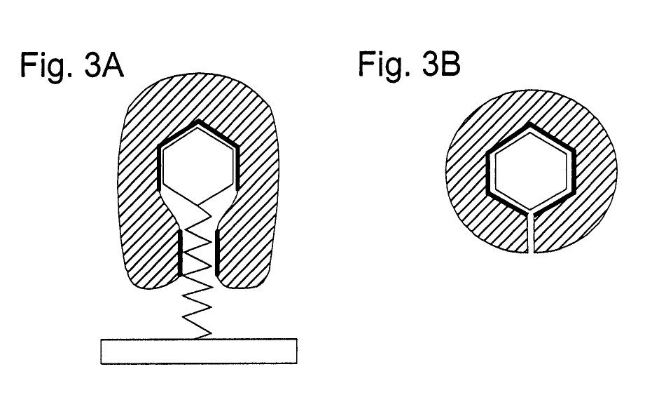

FIG. 2. Schematic representation of the methodology of conventional SELEX.

FIG. 3. Comparison of binding between an aptamer and a target ligand in conventional SELEX (FIG. 3A), where the ligand is attached to a stationary support, and CE-SELEX (FIG. 3B), where binding takes place in the solution phase. The thicker lines indicate binding surfaces on the aptamer.

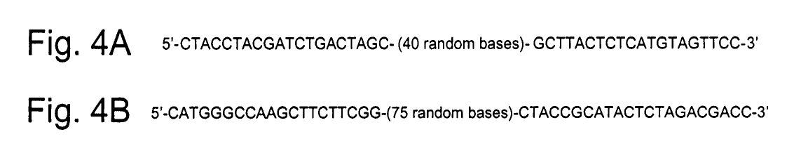

FIG. 4. Sequences to be used in the initial DNA pool of the CE-SELEX selection for aptamers with affinity for IgE (FIG. 4A), and ATP (FIG. 4B).

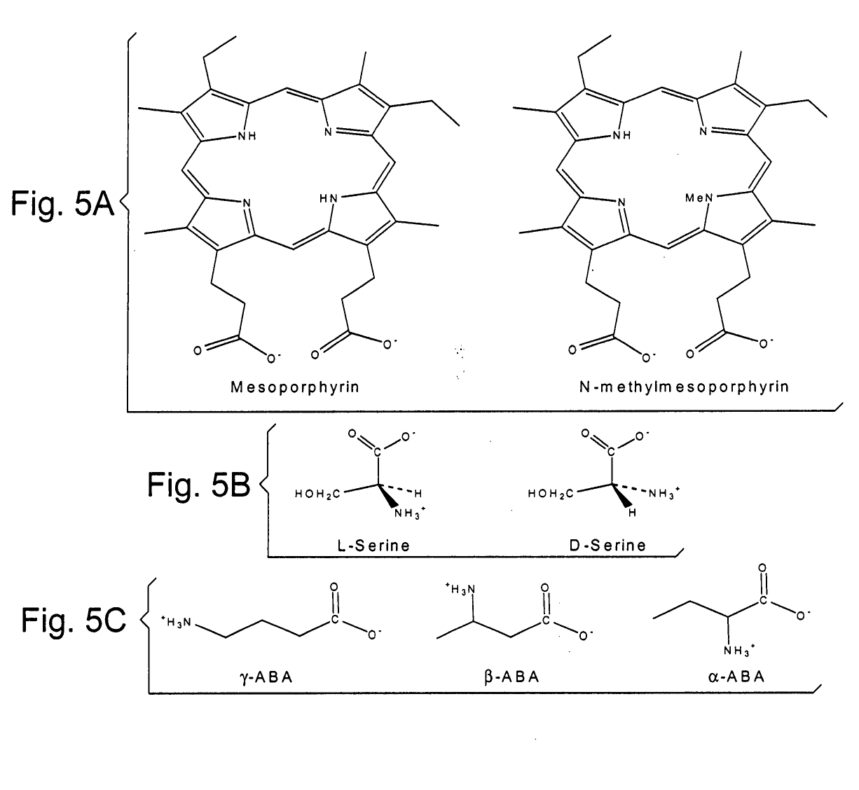

FIG. 5. Structures of the targets to be used to test if CE-SELEX yields aptamers with improved selectivity. CE-SELEX selections will be performed to obtain aptamers selective for N-methylmesoporphyrin over Mesoporphyrin (FIG. 5A), D-serine over L-serine (FIG. 5B), and γ-aminobutyric acid over β-aminobutyric acid and α-aminobutyric acid (FIG. 5C).

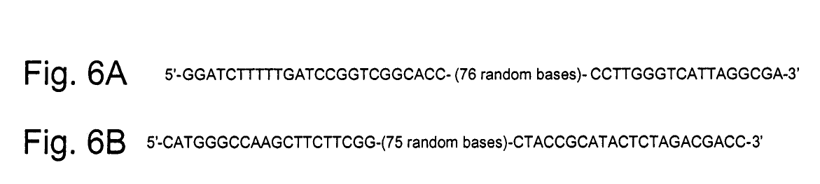

FIG. 6. Sequences to be used in the initial DNA pool of the CE-SELEX selection for aptamers with affinity for NMM (FIG. 6A) and D-serine or γ-ABA (FIG. 6B).

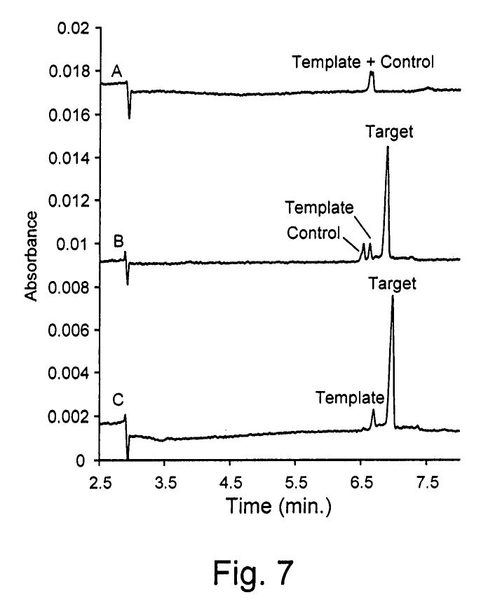

FIG. 7. Electropherograms of 2 μM Template+2 μM Control (FIG. 7A), 2 μM Template+2 μM Control+80 μM Target (FIG. 7B), and 2 μM Template+80 μM Target (FIG. 7C).

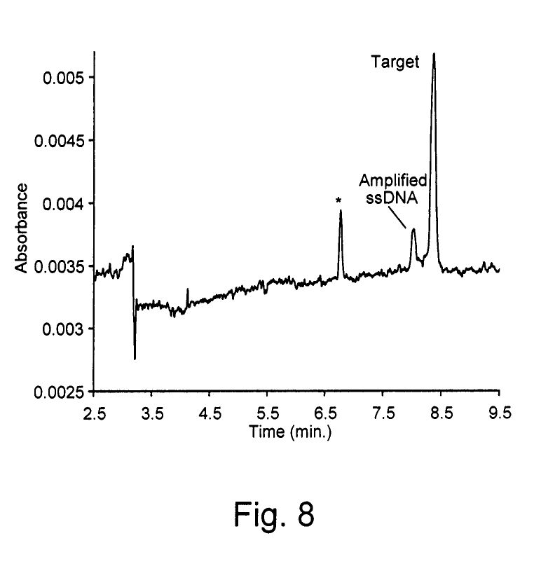

FIG. 8. Electropherogram of 4 μL of the single stranded PCR products spiked with 1 μL 200 μM Target. The ssDNA concentration was estimated to be 1.9 μM. The peak labeled with an asterisk is an unidentified contaminant from the PCR or the clean-up.

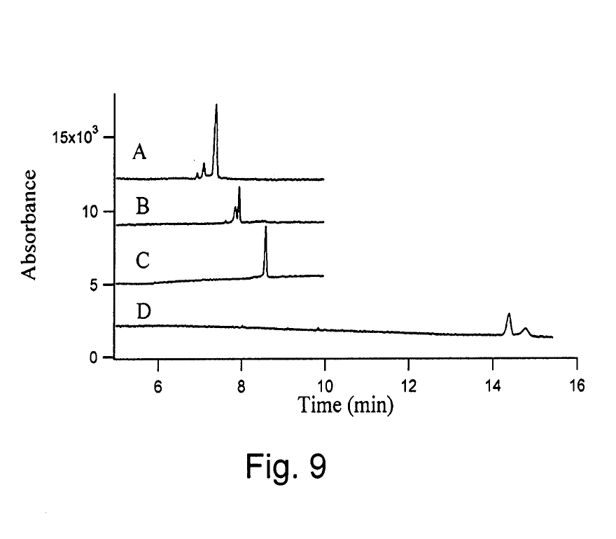

FIG. 9. Electropherograms of 2 μM Template+80 μM Target with no NaCl (FIG. 9A), 2 μM Template+30 μM Target with 10 mM NaCl (FIG. 9B), 2 μM Template+30 μM Target with 20 mM NaCl (FIG. 9C), and 2 μM Template+15 μM Target with 30 mM NaCl (FIG. 9D).

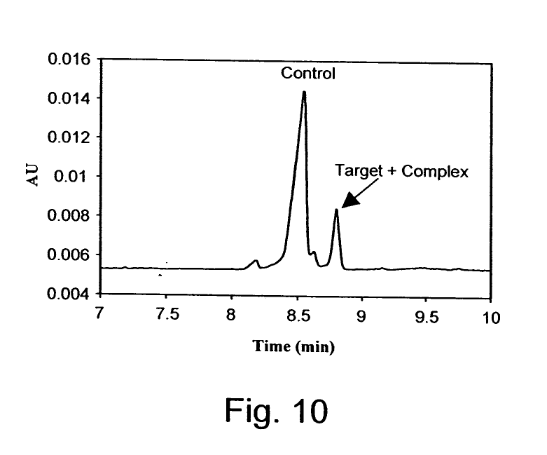

FIG. 10. Electropherogram of 100 μM control, 2 μM template, and 30 μM target.

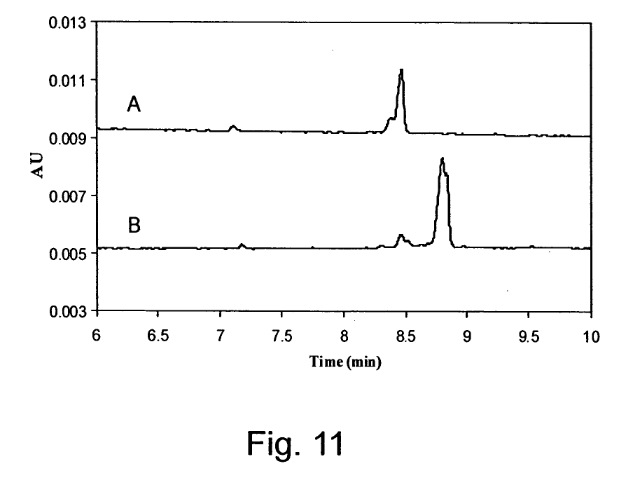

FIG. 11. Electropherograms of the single stranded PCR products from the end of the second CE-SELEX cycle (FIG. 11A) and the single stranded PCR products from the end of the second CE-SELEX cycle spiked with 20 mM of the target (FIG. 11B).

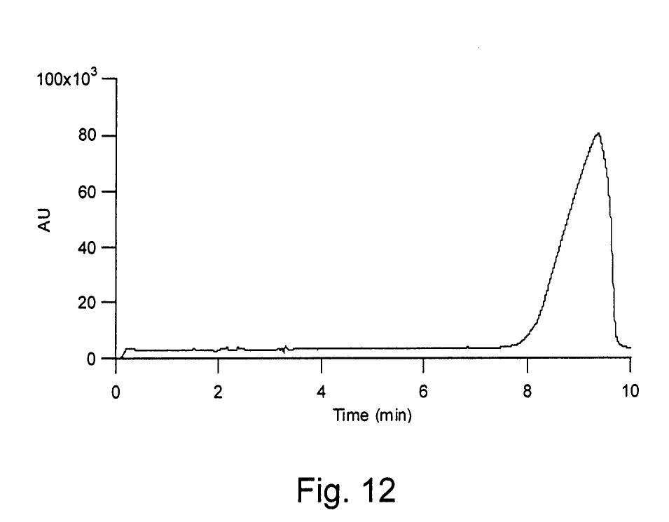

FIG. 12. Electropherogram of 1 mM library and 5 μM target.

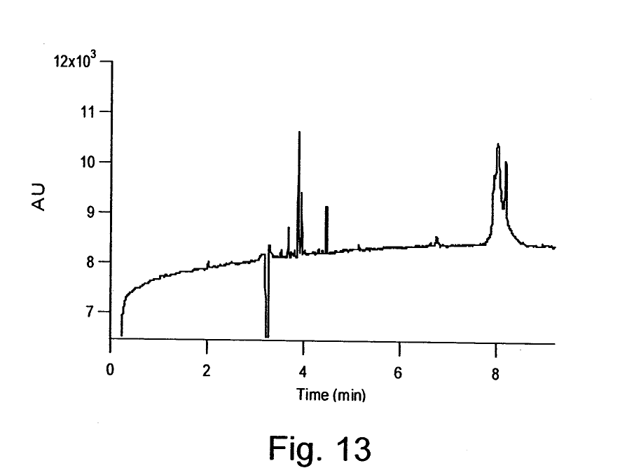

FIG. 13. Electropherograms of the single stranded PCR products from the end of the second CE-SELEX cycle.

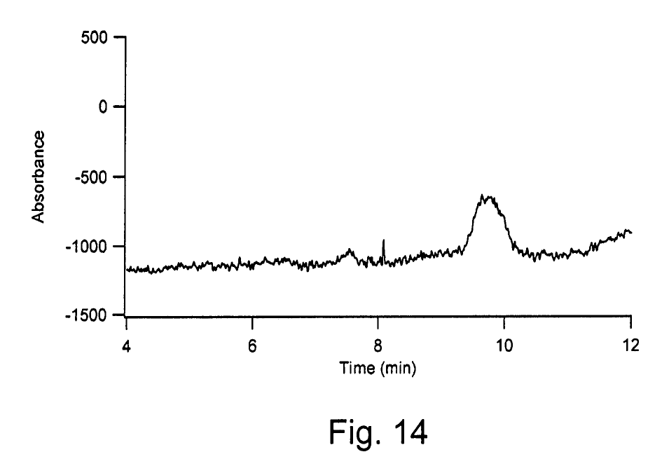

FIG. 14. Electropherogram of ssDNA+80 μM target.

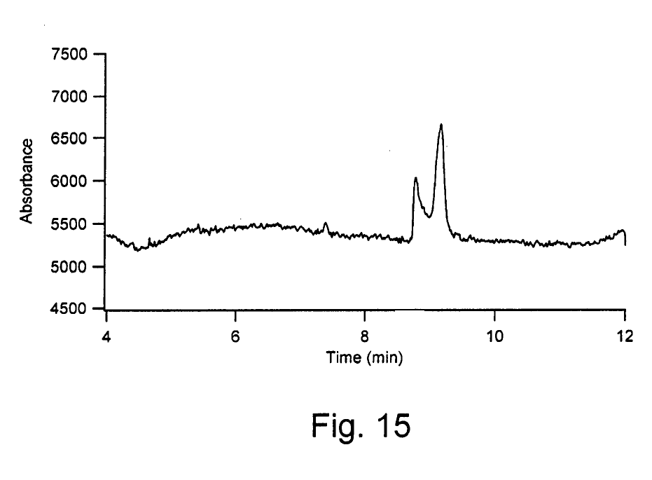

FIG. 15. Electropherogram of ssDNA+30 μM target.

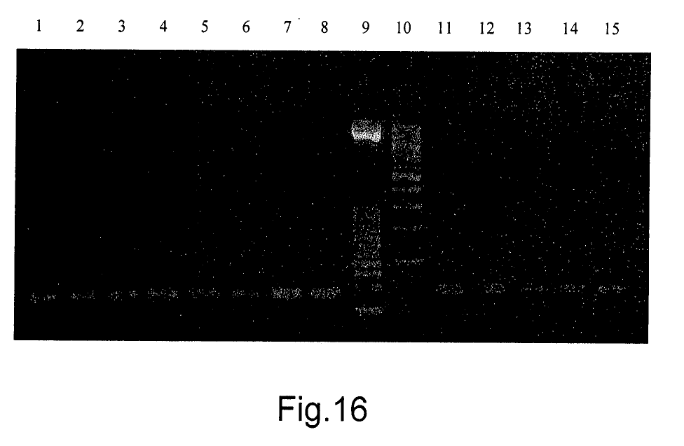

FIG. 16. Gel electrophoresis analysis of PCR products at different MgCl₂ concentrations. The lane assignments are as follows: lane 1—1,000 starting DNA molecules and 4.5 mM MgCl₂; lane 2—1,000,000 starting DNA molecules and 4.5 mM MgCl₂; lane 3—Control PCR with 4.5 mM MgCl₂; lane 4—1,000,000 starting DNA molecules and 6.0 mM MgCl₂; lane 5—Control PCR with 6.0 mM MgCl₂; lane 6—1,000 starting DNA molecules and 7.5 mM MgCl₂; lane 7—1,000,000 starting DNA molecules and 7.5 mM MgCl₂; lane 8—Control PCR with 7.5 mM MgCl₂; lane 9—25 bp DNA step ladder; lane 10—100 bp DNA step ladder; lane 11—1,000,000 starting DNA molecules and 9.0 mM MgCl₂; lane 12—Control PCR with 9.0 mM MgCl₂; lane 13—1,000 starting DNA molecules and 10.5 mM MgCl₂; lane 14—1,000,000 starting DNA molecules and 10.5 mM MgCl₂; lane 15—Control PCR with 10.5 mM MgCl₂.

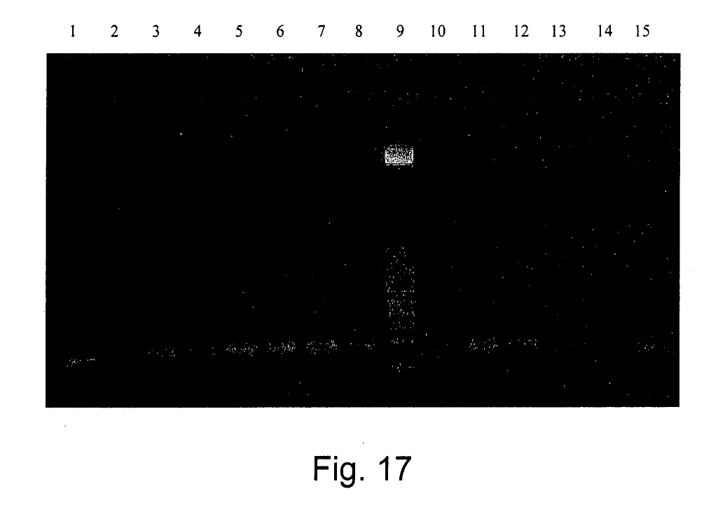

FIG. 17. Gel electrophoresis analysis of PCR products using an annealing temperature of 46° C. and different MgCl₂ concentrations. Lane assignments are as follows: lane 1—1,000 starting DNA molecules and 4.5 mM MgCl₂; lane 2—1,000,000 starting DNA molecules and 4.5 mM MgCl₂; lane 3—Control PCR with 4.5 mM MgCl₂; lane 4—1,000,000 starting DNA molecules and 6.0 mM MgCl₂; lane 5—Control PCR with 6.0 mM MgCl₂; lane 6—1,000 starting DNA molecules and 7.5 mM MgCl₂; lane 7—1,000,000 starting DNA molecules and 7.5 mM MgCl₂; lane 8—Control PCR with 7.5 mM MgCl₂; lane 9—25 bp DNA step ladder; lane 10—empty lane; lane 11—1,000,000 starting DNA molecules and 9.0 mM MgCl₂; lane 12—Control PCR with 9.0 mM MgCl₂; lane 13—1,000 starting DNA molecules and 10.5 mM MgCl₂; lane 14—1,000,000 starting DNA molecules and 10.5 mM MgCl₂; lane 15—Control PCR with 10.5 mM MgCl₂.

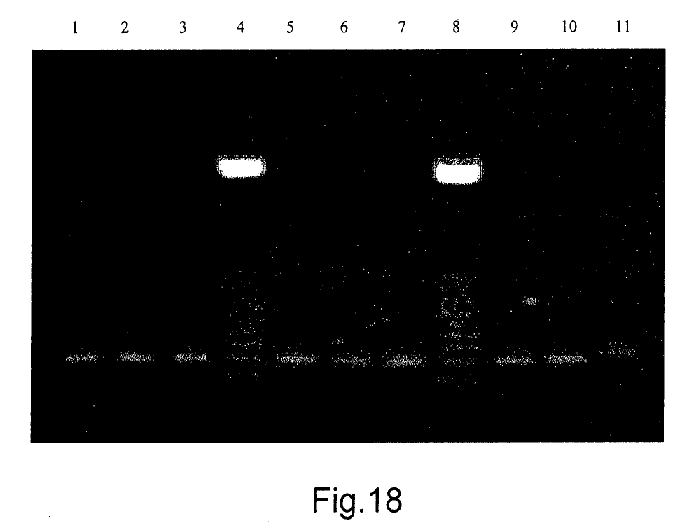

FIG. 18. Gel electrophoresis analysis of PCR products from a CE fraction of the template DNA with new PCR primers. Lane assignments are as follows: lanes 1 to 3, 5 to 7, 9, and 10 are PCR samples containing the template DNA; lanes 4 and 8 contain the 25 bp DNA step ladder, and lane 11 contains the PCR control reaction.

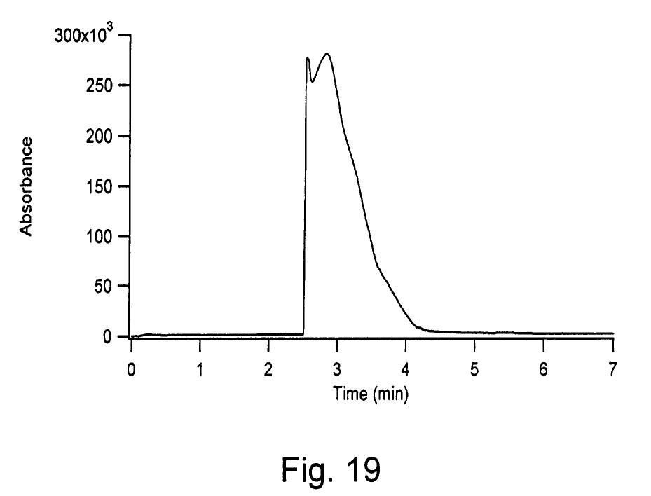

FIG. 19. Electropherogram of the first CE-SELEX round of Example 10 with 1.6 mM DNA library and 1 μM IgE.

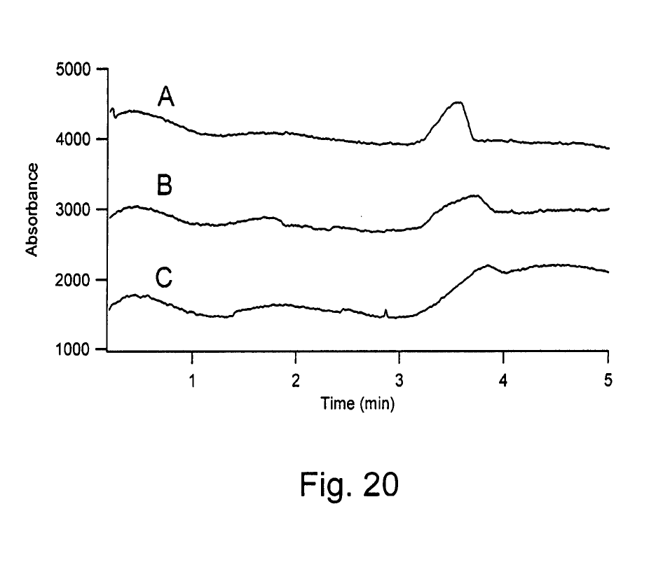

FIG. 20. Electropherograms of the binding assay of the first CE-SELEX round for the first experiment of Example 10. 1 μL ssDNA+0 μM IgE (FIG. 20A), 1 μL ssDNA+1.5 μM IgE (FIG. 20B), and 1 μL ssDNA+3 μM IgE (FIG. 20C).

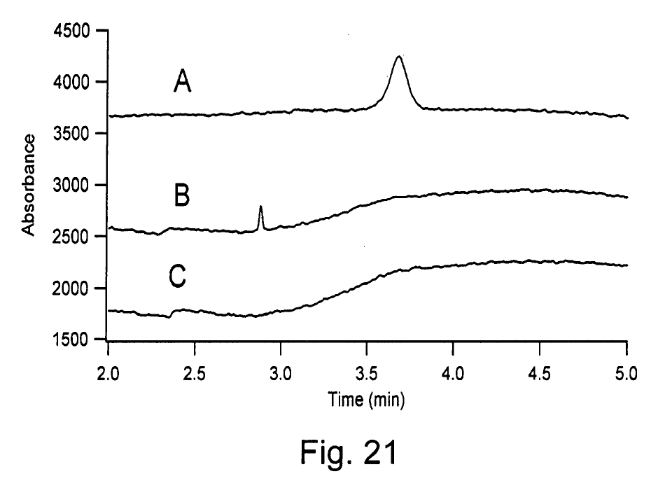

FIG. 21. Electropherograms of the binding assay of the second CE-SELEX round for the first experiment. 1 μL ssDNA+0 μM IgE (FIG. 21A), 1 μL ssDNA+1.5 μM IgE (FIG. 21B), and 1 μL ssDNA+2.5 μM IgE (FIG. 21C). The small peak in B is from an unidentified contaminant.

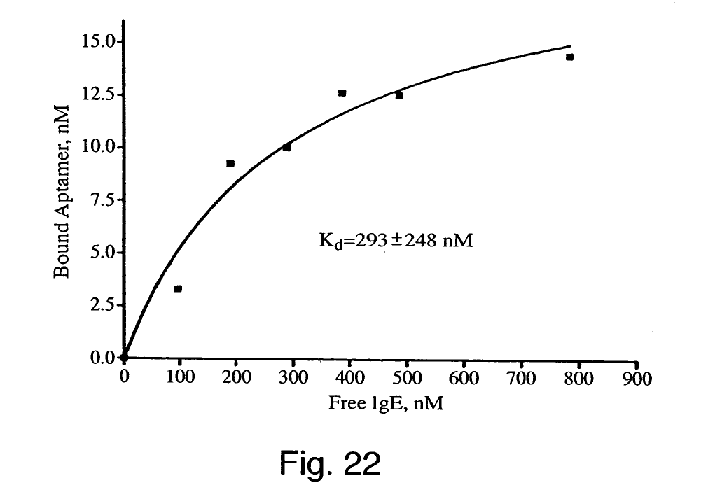

FIG. 22. Binding curve with results from the non-linear regression analysis for Clone 1.8.

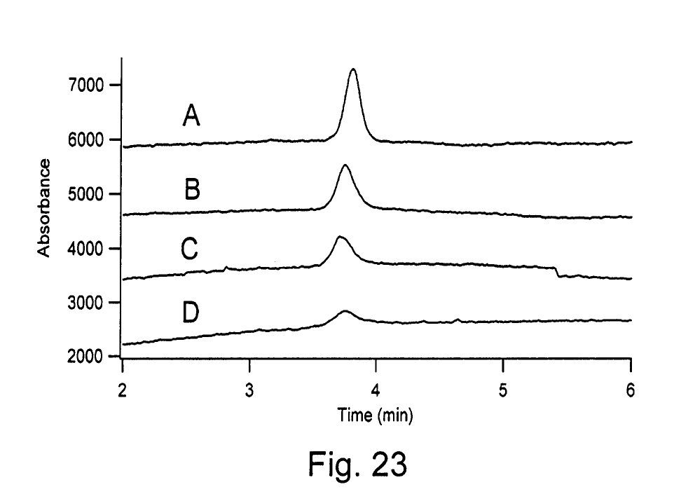

FIG. 23. Electropherograms of the binding assay of the first CE-SELEX round for the second experiment. 1 μL ssDNA+0 μM IgE (FIG. 23A), 1 μL ssDNA+0.4 μM IgE (FIG. 23B), 1 μL ssDNA+1.1 μM IgE (FIG. 23C), and 1 μL ssDNA+2.3 μM IgE (FIG. 23D).

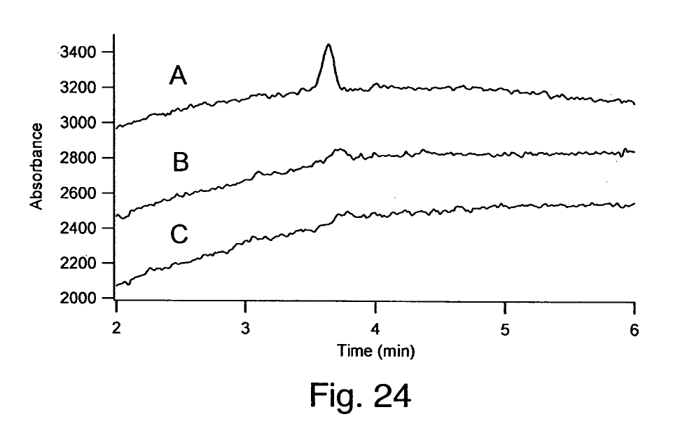

FIG. 24. Electropherograms of the binding assay of the second CE-SELEX round for the second experiment. 1 μL ssDNA+0 μM IgE (FIG. 24A), 1 μL ssDNA+0.4 μM IgE (FIG. 24B), and 1 μL ssDNA+1.1 μM IgE (FIG. 24C).

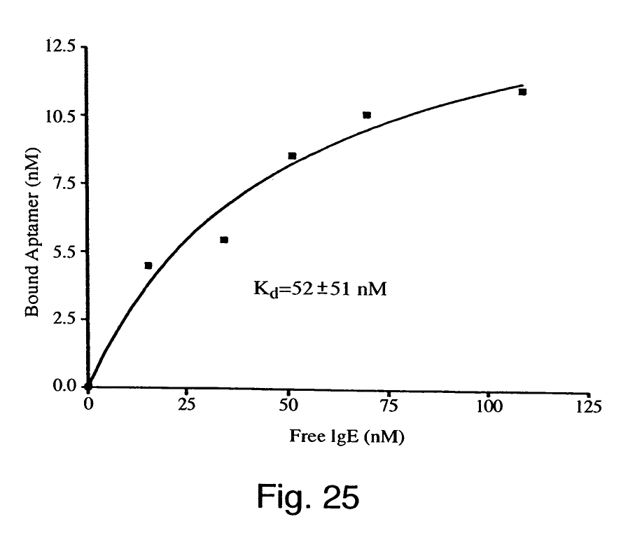

FIG. 25. Binding curve with results from the non-linear regression analysis for Clone 2.27.

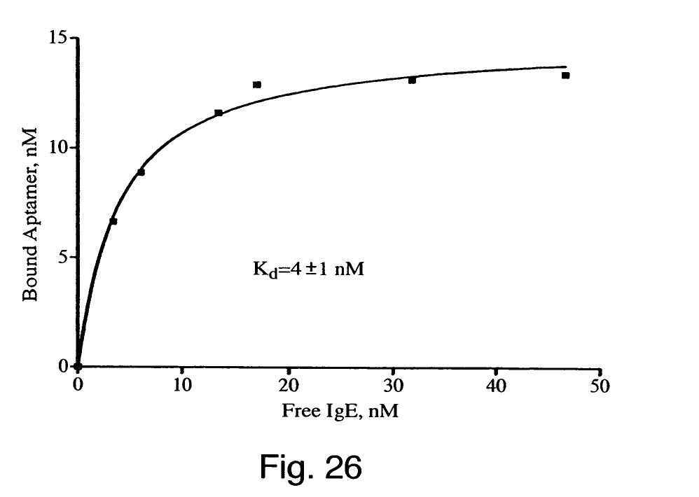

FIG. 26. Binding curve with results from the non-linear regression analysis for the conventional SELEX aptamer.

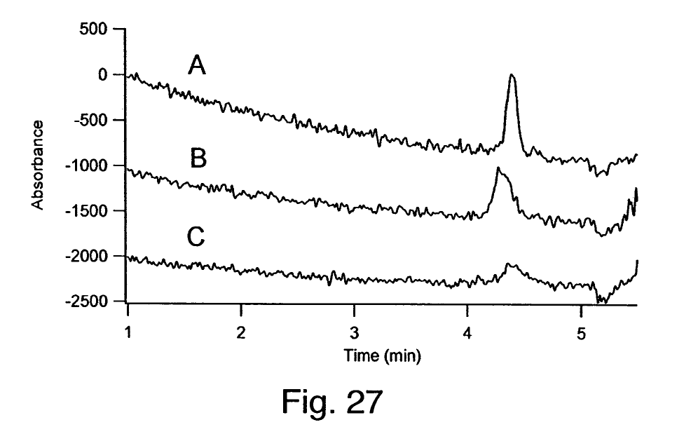

FIG. 27. Electropherograms of the binding assay of the first CE-SELEX round for the third experiment. 0.5 μL ssDNA+0 μM IgE (FIG. 27A), 0.5 μL ssDNA+0.3 μM IgE (FIG. 27B), and 0.5 μL ssDNA+0.8 μM IgE (FIG. 27C).

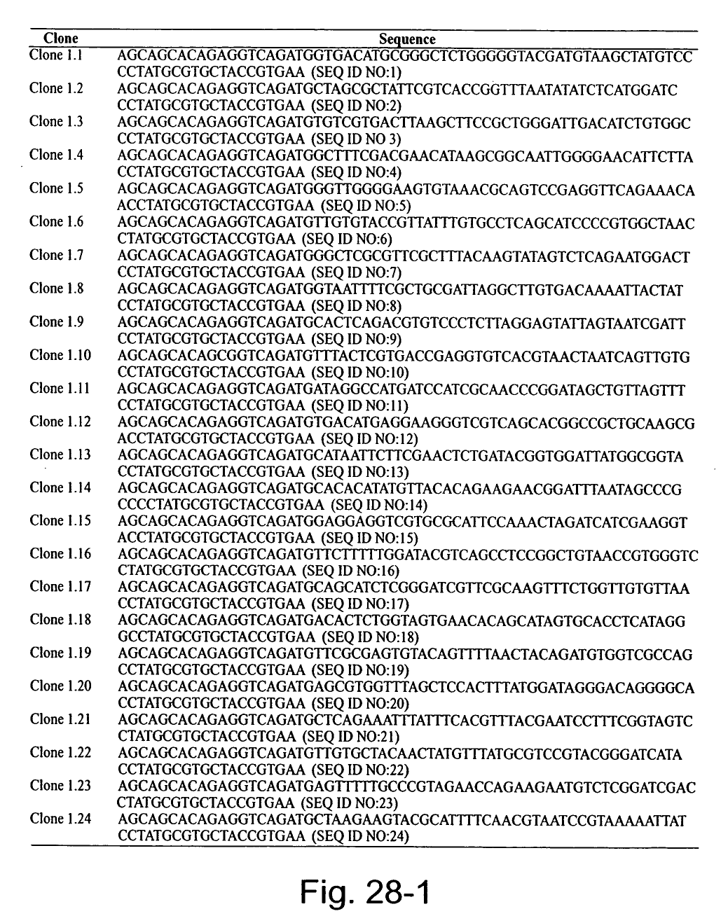

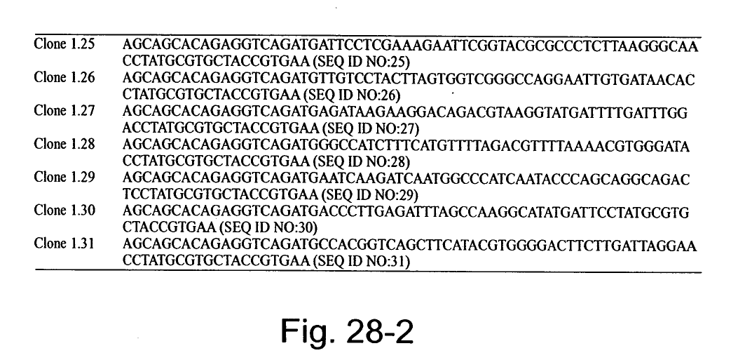

FIG. 28. Sequences of the clones from the first CE-SELEX experiment.

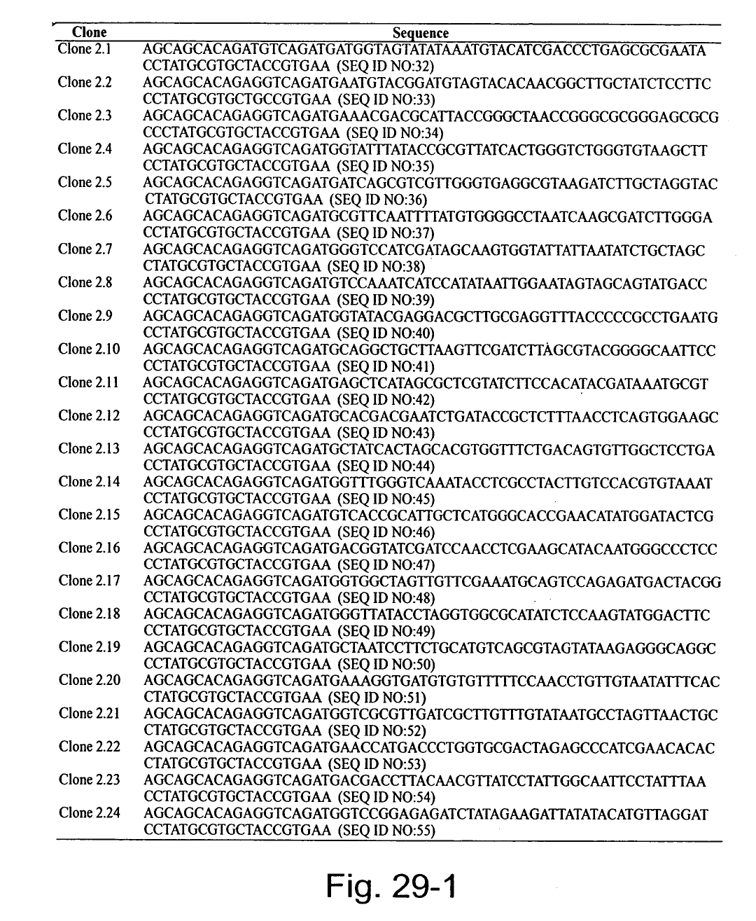

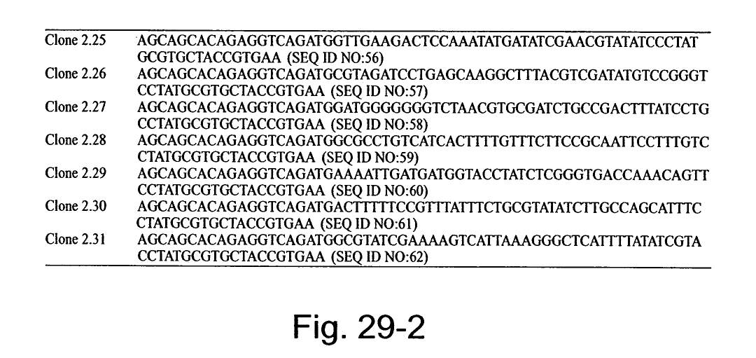

FIG. 29. Sequences of the clones from the second CE-SELEX experiment.

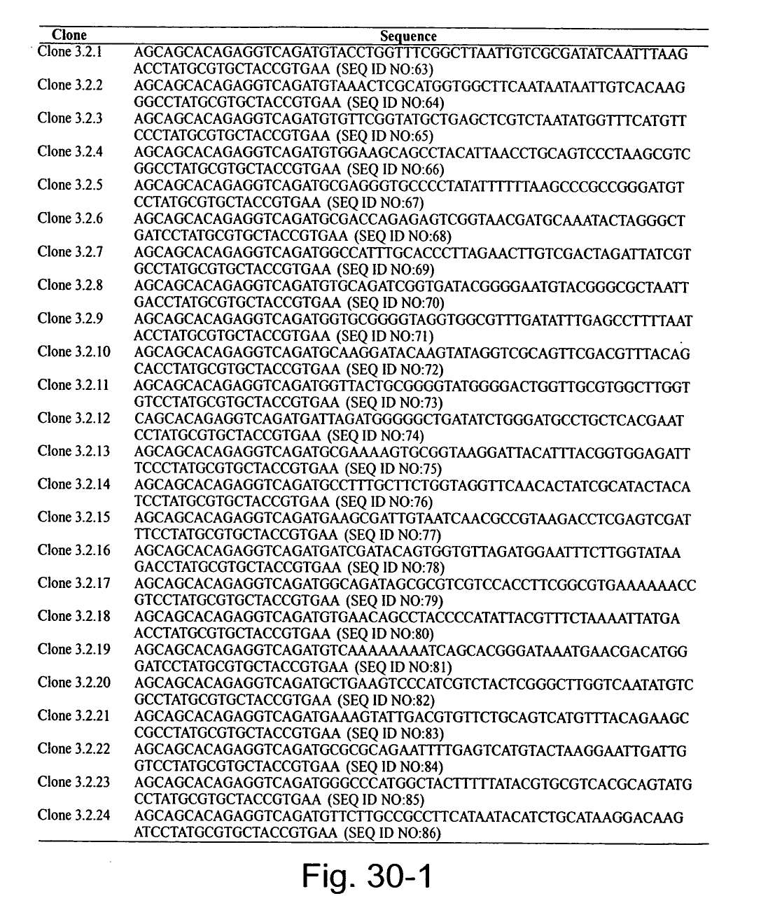

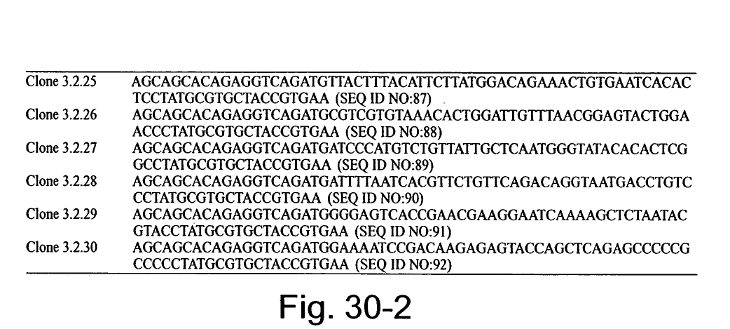

FIG. 30. Sequences of the clones from the second round of the third CE-SELEX experiment.

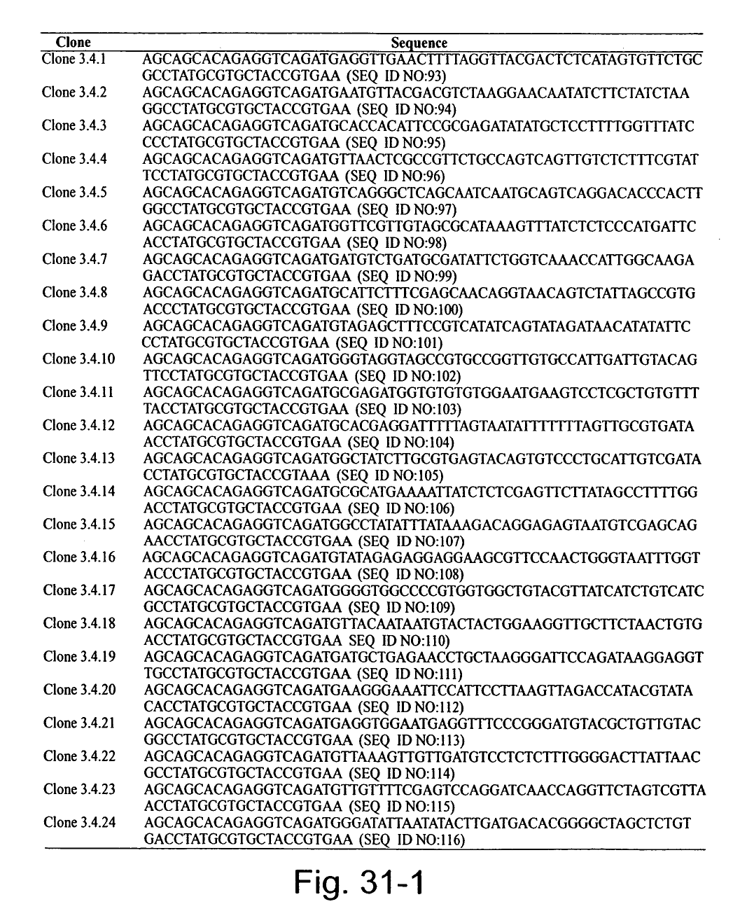

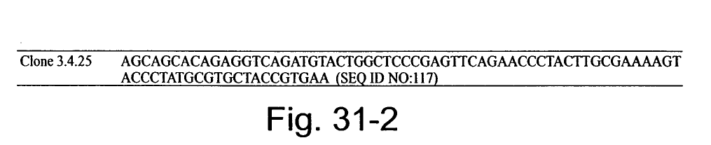

FIG. 31. Sequences of the clones from the fourth round of the third CE-SELEX experiment.

### DETAILED DESCRIPTION OF PREFERRED EMBODIMENTS

CE-SELEX is a novel selection procedure that utilizes capillary electrophoresis (CE) in combination with conventional SELEX selection procedures. CE-SELEX, for the first time, allows selection to be performed in free solution. Performing selection against a target in free solution can yield aptamers with higher affinity and/or higher selectivity toward target molecules. Improving selection procedures is important since aptamers with improved affinity and selectivity will demonstrate increased activity when used as pharmaceuticals or diagnostic agents.

SELEX, an acronym for “Systematic Evolution of Ligands by EXponential enrichment,” was independently developed by the research groups of G. F. Joyce (see Joyce, Gene, 1989; 82:83-87), J. W. Szostak (see Ellington et al., Nature, 1990; 346:818-822), and L. Gold (see Tuerk et al., Science, 1990; 249:505-510). SELEX makes use of evolution on a molecular scale to select functional sequences of DNA or RNA (also called “aptamers”) from random combinatorial libraries. Several reviews describing SELEX have recently been published (Joyce, Curr. Opin. Struct. Biol., 1994; 4:331-336); Lorsch et al., Acc. Chem. Res., 1996; 29:103-110; Gold et al., Annu. Rev. Biochem., 1995; 64:763-797; Forst, J. Biotech., 1998; 64:101-118; Klug et al., Mol. Biol. Rep., 1994; 20:97-107; Osborne et al., Chem. Rev., 1997; 97:349-370) and U.S. Pat. Nos. 5,270,163, 5,475,096, and 5,707,796 also describe the conventional SELEX technology. The general procedure for conventional SELEX is shown in FIG. 2. A pool of randomly sequenced DNA is generated (approximately 10¹⁴-10¹⁵ independent sequences). Often the DNA is transcribed to RNA, which has been shown to be more functional than DNA. The RNA pool is then passed through an affinity column with the target molecule attached to the stationary phase. RNAs with affinity toward the immobilized target molecule are retained on the column. RNAs with little or no affinity for the target molecule are washed off the column to waste. The bound RNAs are then eluted off the column using a solution containing the free ligand and are reverse transcribed. The DNA can then be amplified using the polymerase chain reaction (PCR). When repeated several times, the selection cycle eliminates the inactive RNAs from the pool, leaving only sequences with affinity for the target molecule. As shown in Table 1, the SELEX procedure has been successful in selecting molecules that have affinity for various target molecules; including IgE (Wiegand et al., J. Immun., 1996; 157:221-230) and ATP (Huizenga et al., Biochem., 1995; 34:656-665).

TABLE 1
Dissociation Constants (K_{D}) of aptamers selected to bind various target
molecules.

| Target Ligand | Pool | K_{D} (nM) |
|---|---|---|
| Large Molecules | | |
| E. coli metJ protein¹ | DNA | 1 |
| Elastase^{2, 3} | DNA | 7 |
| | Non-standard Base | 15 |
| HIV-1 RT⁴ | DNA | 7 |
| IgE⁵ | DNA | 10 |
| | Non-standard Base | 30 |
| Lrp⁶ | DNA | 2 |
| Thrombin^{7, 8} | DNA | 25 |
| | Non-standard Base | 400 |
| Small Molecules | | |
| L-arginine⁹ | RNA | 10,000 |
| D-arginine¹⁰ | RNA | 200,000 |
| ATP^{11, 12, 13} | DNA | 6,000 |
| | DNA | 9 × 10⁻⁶ M^{2 a} |
| | RNA | 700 |
| | Non-standard Base | 6 × 10⁻⁶ M^{2 a} |
| Kanamycin¹⁴ | RNA | ≈300 |
| Lividomycin¹⁴ | RNA | ≈300 |
| neomycin¹⁵ | RNA | 100 |
| nicotinamide (NAD)¹⁶ | RNA | 2,500 |
| N-methylmesoporphyrin IX¹⁷ | DNA | 500 |
| Theophylline¹⁸ | RNA | 320 |
| Tobramycin¹⁹ | RNA | 6 |
| D-tryptophan²⁰ | RNA | 1,800 |
| L-valine²¹ | RNA | 2,900,000 |
| Vitamin B12²² | RNA | 88 |

^{a}The binding stoichiometry was found to be 2:1 (ATP:aptamer). One-half of the aptamer is bound at ATP concentrations of 3,000,000 and 2,400,000 nM for the DNA and modified base aptamers, respectively.
¹He et al., J. Mol. Biol., 1996; 255:55-66.
²Lin et al., Proc. Natl. Acad. Sci., 1995; 92:11044-11048.
³Lin et al., Nucleic Acids Res., 1994; 22:5229-5234.
⁴Schneider et al., Biochem., 1995; 34:9599-9610.
⁵Wiegand et al., J. Immun., 1996; 157:221-230.
⁶Cui et al., J. Bacteriology, 1995; 177:4872-4880.
⁷Bock et al., Nature, 1992; 355:564-566.
⁸Latham et al., Nucl. Acids Res., 1994; 22:2817-2822.
⁹Famulok, J. Am. Chem. Soc., 1994; 116:1698-1706.
¹⁰Connell et al., Biochem., 1993; 32:5497-5502.
¹¹Huizenga et al., Biochem., 1995; 34:656-665.
¹²Battersby et al., J. Amer. Chem. Soc., 1999; 121:9781-9789.
¹³Sassanfar et al., Nature, 1993; 364:550-553.
¹⁴Lato et al., Chem. Biol., 1995; 1995:291-303.
¹⁵Wallis et al., Chem. Biol., 1995; 2:543-552.
¹⁶Lauhon et al., J. Amer. Chem. Soc., 1995; 117:1246-1257.
¹⁷Li et al., Biochem., 1996; 35:6911-6922.
¹⁸Jenison et al., Science, 1994; 263:1425-1429.
¹⁹Wang et al., Chem. Biol., 1995; 2:281-290.
²⁰Famulok et al., J. Am. Chem. Soc., 1992; 114:3990-3991.
²¹Majerfeld et al., Nature Struct. Biol., 1994; 1:287-292.
²²Lorsch et al., Biochem., 1994; 33:973-982.

SELEX is an “evolutionary” approach to combinatorial chemistry that uses in vitro selection to identify RNA or DNA sequences with affinity for a particular target. For this reason, the procedure is also known as “in vitro selection” or “in vitro evolution.” The process separates functional molecules from random DNA or RNA pools using affinity chromatography. DNA or RNA sequences that demonstrate affinity for the target are amplified using PCR, mutated and reselected against the target. Several repetitions of this cycle results in a pool of DNA or RNA sequences with affinity for the target molecule. These functional sequences, also referred to as aptamers, have found use as drugs that act on specific biological receptors or as diagnostic agents that can be used in biomedical analyses or imaging.

However, as the initial excitement over SELEX selection has began to fade, several researchers have raised troubling questions about conventional SELEX selection methods. For example, as shown in Table 1, in vitro selection often produces aptamers with nanomolar dissociation constants for most large ligands (e.g. proteins). Why have sequences with stronger binding not been discovered? Is the binding strength of selected molecules limited by the limited functionality of the RNA and DNA pool or is there some flaw in the selection procedure? Several studies, in which the researchers attempted to add functionality to DNA through the use of non-standard bases, have produced aptamers with binding efficiencies not substantially different from those selected from a standard DNA pool (see Table 1). This suggests that the selection procedure, not the functionality of the DNA (or RNA) limits the binding effectiveness of the selected molecule. Further, in vitro selection is much poorer when smaller ligands are used, with dissociation constants typically in the micromolar range (see Table 1).

Upon reflection, it is clear that the selection procedure used in conventional SELEX is flawed. The problem with the conventional selection process is that the aptamer is selected to have affinity for the target molecule bound to a stationary support, not free in solution. The evolutionary process, which is the strength of in vitro selection, now works against the researcher. Instead of converging on an aptamer that has affinity for the desired target, the process selects an aptamer that binds a molecule similar to the target. Koizumi and Breaker have performed experiments that support this premise, demonstrating that aptamers selected to bind cAMP actually had stronger affinity for cAMP analogs modified at the C8 position, the same position where the target was tethered to the stationary support (Koizumi et al., Biochem., 2000; 39:8983-8992). The “optimal” binding sequence is limited by the selection procedure, not the functionality of the RNA or DNA pool, explaining both why aptamers with picomolar dissociation constants have not been discovered and why the introduction of non-standard bases has had limited effect.

The effect of the stationary support is much greater when selecting for smaller ligands. Small ligands only have a limited number of functionalities that can interact with the aptamer. Attaching the ligand to a stationary support removes one of these functionalities. Also, tightest binding with an RNA or DNA molecule would likely occur when the aptamer can completely wrap around the ligand, interacting with all of the available binding sites (see FIG. 3). The stationary support prevents this, introducing bias against the sequences that would be expected to bind the ligand best.

Other problems are introduced by the stationary support. Some researchers have suggested that the rinsing step where the active sequences are removed from the column with a solution of free ligand may actually bias against aptamers with very high affinity for the ligand (Klug et al., Mol. Biol. Rep., 1994; 20:97-107). Sequences with high affinity for the ligand would not wash off the column easily. Therefore, it may be impossible to recover sequences with picomolar or lower dissociation constants from the selection column. A recent study has demonstrated aptamers with strongly co-operative binding for two ligands (Battersby et al., J. Amer. Chem. Soc., 1999; 121:9781-9789). This was not expected and is likely the result of an anomaly introduced by selecting for ligand bound to a stationary support.

Conventional SELEX makes use of chromatography to perform this separation and because of the evolutionary biases introduced by the stationary phase, is perhaps not the one best separation technology suited for SELEX. The most obvious bias introduced by chromatographic separation is that selection is not performed against the actual target. Instead a sequence is selected to have affinity for the target attached to a stationary support. Another concern is kinetic bias where it is almost impossible to elute very strongly interacting sequences from a chromatography column. Obviously, it would be desirable to select an aptamer with binding affinity for the actual target molecule, not the ligand bound to some stationary support. Unfortunately no studies have been published where selection has been attempted using an unbound target molecule.

CE-SELEX:

CE-SELEX of the present invention is a novel selection procedure that utilizes capillary electrophoresis (CE) in combination with conventional SELEX selection. The CE-SELEX procedure provides significant advantages over the conventional SELEX procedure. The most important advantage is that molecules are selected to interact with the target in free solution. This is preferable to selecting RNAs or DNAs that interact with the target attached to a stationary support. Selecting against the actual target in the solution phase will result in aptamers with higher affinity for the target molecule and better selectivity against similar analytes. This improvement will be especially true for small molecules. There will be no need to wash the RNAs or DNAs off the column and therefore no kinetic discrimination against molecules that interact strongly with the target as in conventional SELEX. Further, it will be easier to vary the selection conditions in CE-SELEX. The selection criteria can be adjusted in CE-SELEX by simply varying the concentration of the target in the separation buffer. In conventional SELEX a new column with a different stationary phase needs to be prepared. Being able to vary the separation conditions will also make it easier to mimic biological conditions.

CE-SELEX is a method for identifying nucleic acid ligands to a target from a candidate mixture of nucleic acids. The method includes the steps of contacting a candidate mixture of nucleic acids with the target molecule, partitioning between members of the candidate mixture with an increased affinity for the target and the remainder of the candidate mixture by capillary electrophoresis; and amplifying, in vitro, the selected members of the candidate mixture with increased affinity for the target, to yield a ligand-enriched mixture of nucleic acids, whereby nucleic acid ligands of the target molecule may be identified. The steps of contacting, partitioning, and amplifying may be performed only once, as a single cycle, to yield a ligand-enriched mixture of nucleic acids. Or, the steps of contacting, partitioning, and amplifying may be repeated for several or many cycles, to yield a ligand-enriched mixture of nucleic acids. As few as two cycles, or as many as twenty cycles, may be performed. For example, 2, 3, 4, 5, 6, 7, 8, 9, 10, 11, 12, 13, 14, 15, 16, 17, 18, 19, or 20 cycles may be performed. The number of cycles may include a range of any of the above numbers. For example, the number of cycles may include, but is not limited to, 2-5 cycles, 2-10 cycles, 2-20 cycles, 5-10 cycles, 5-15 cycles, 8-10 cycles, or 8-12 cycles.

The CE-SELEX procedure of the present invention will facilitate automation of the SELEX process. Conventional SELEX is labor intensive and not particularly compatible with large-scale automation. CE-SELEX will allow numerous experiments to be performed in parallel. Capillary electrophoresis has been demonstrated in a 96 capillary array format (Pang et al., J. Biochem. Biophys. Methods, 1999; 41:121-132) that provides direct compatibility with the standard 96 well PCR plate. Another use of CE-SELEX includes the integration of the SELEX process into a microfluidic chip, as it is now possible to design a chip that performs electrophoretic separations (Colyer et al., J. Chrom. A, 1997; 781:271-276; Jacobson et al., Anal. Chem., 1998; 70:3476-3480), immunoassays (Chiem et al., Clin. Chem., 1998; 44:591-598; (Chiem et al., Electrophoresis, 1998; 19:3040-3044), online reactions (Jacobson et al., Anal. Chem., 1994; 66:4127-4132; Fluri et al., Anal. Chem., 1996; 68:4285-4290), or even PCR (Cheng et al., Anal. Biochem., 1998; 257:101-106; Waters et al., Anal. Chem., 1998; 70:5172-5176). Thus, it will be possible to perform all of the operations of CE-SELEX on a single chip.

Capillary Electrophoresis (CE):

CE-SELEX makes use of capillary electrophoresis (CE). CE provides fast, high efficiency separations based on the size and charge of an analyte in the solution phase (Landers, Ed., Handbook of Capillary Electrophoresis, 1997; CRC Press, New York). It is well known that sequence and length have minimal effect on ssDNA (or RNA) mobility in free solution (Stellwagen et al., Biopolymers, 1997; 41:687-703; Righetti et al., J. Biochem. Biophys. Methods, 1999; 41:75-90; Hoagland et al., Macromol., 1999; 32:6180-6190), forcing CE based sequencing to be performed in capillary gels. Stellwagen et al. demonstrated that mobility changed <5% over a range of 30-412 bp ssDNA (Stellwagen et al., Biopolymers, 1997; 41:687-703). Similarly Hoagland et al. measured only a 2% mobility difference between 15 and >650 bp poly(dT) (Hoagland et al., Macromol., 1999; 32:6180-6190). Perego et al. have shown that relatively extreme conditions (pH=3.3) were necessary for differences in ssDNA sequence to contribute to mobility (Perego et al., Electrophoresis, 1997; 18:2915-2920). This effect was only significant for DNAs shorter than 60 bp.

Contrary to the case in sequencing, the co-migration of RNAs (or DNAs) is beneficial for CE-SELEX selection. In the absence of the target all RNAs or DNAs migrate through the capillary in a single zone. If a target molecule is added to the separation buffer, the RNAs (or DNAs) that interact with the target will migrate at a different velocity than the non-interacting molecules, forming a second zone. The two zones can be collected into different vials, thereby separating the active sequences from the inactive ones. FIG. 1 shows how CE is incorporated into the SELEX process. It is also well known that molecular interactions affect analyte mobilities in CE in a predicable manner (Chu et al., Acc. Chem. Res., 1995; 28:461-468; Rippel et al., Electrophoresis, 1997; 18:2175-2183; Colton et al., Electrophoresis, 1998; 19:367-382; Heegaard et al., J. Chrom., 1998; B 715:29-54).

CE selections for the present invention may be performed on any commercially available CE instrument to maximize migration time reproducibility. CE selections for the present invention may also be performed on CE instruments that have been built in house. Such instruments may or may not be equipped with a liquid cooling system to minimize the effects of Joule heating.

Coated or uncoated capillaries may be used. Coated capillaries may be used to improve migration time reproducibility and minimize interactions between the target molecules and the fused silica wall.

Capillaries may be filled with a selection buffer, also referred to as “CE selection buffer,” “CE run buffer,” or “CE buffer.” CE buffer may be similar to that used for conventional SELEX experiments. As needed, the CE buffer used for CE-SELEX may be varied or optimized. For example, the pH or ionic strength may be modified. For example, the salt concentration of CE buffer may be varied from about 0 mM NaCl to about 50 mM NaCl, including for example, a salt concentration of about 0 mM NaCl, about 5 mM NaCl, about 10 mM NaCl, about 15 mM NaCl, about 20 mM NaCl, about 25 mM NaCl, about 30 mM NaCl, about 35 mM NaCl, about 40 mM NaCl, about 45 mM NaCl, and about 50 mM NaCl. The salt concentration of CE buffer may include a range of any of the above concentrations. Other modifications that may be made to the CE buffer include, but are not limited to, changing the buffer salt, adding a surfactant, or changing the solvent, to for example, methanol or acetonitrile.

The concentration of the target may be chosen such that it is high enough to ensure there are aptamers present that can bind the target at that concentration but low enough to select for the strongest binders. For example, a plug of the randomized ssDNA solution may be injected onto the head of the separation capillary. Voltage may be applied to migrate the DNA toward the capillary outlet. Interactions with the target molecules cause active (i.e. bound) DNA to migrate at a different velocity than the inactive (i.e., free) DNA. Zones containing DNA that bind the target are collected in different vials than the zones containing non-binding DNA. The zones are collected in vials containing 250 μL aliquots of selection buffer. During the initial rounds of selection the number of active sequences present is expected to be small, making direct detection of the active band impossible. The peak corresponding to the inactive DNA should be readily detectable using UV absorbance detection because of the high DNA concentration necessary.

The following procedure may be used to separate the active sequences from the inactive ones. The outlet end of the capillary is placed in the collection vial at the beginning of the separation to collect any sequences migrating earlier than the inactive DNA. As the inactive DNA begins to migrate off the capillary the outlet is changed to a waste vial. After the inactive DNA has migrated off the column, the outlet is returned to the collection vial. Pressure is used to push any active DNA remaining in the capillary into the collection vial, eliminating the need to wait for very slowly migrating sequences. This procedure does not require any foreknowledge of the migration behavior of the active sequences. The operator does not even have to predict if the target will shift the mobility of the aptamer in the positive or negative direction.

For the best results using the method of the present invention, DNA-wall and target-wall interactions should be reduced, if not eliminated. DNA-wall interactions are typically expected to be minimal. In bare fused silica capillaries charge repulsion between the DNA and the deprotonated silanols on the capillary surface disrupt interactions. Coated capillaries are often designed to minimize interactions with DNA. Target-wall interactions can be more difficult to predict. Previous studies have demonstrated that none of the targets exemplified here exhibit significant wall interactions under the chosen selection conditions (Battersby et al., J. Amer. Chem. Soc., 1999; 121:9781-9789; German et al., Anal. Chem., 1998; 70:4540-4545). Thus, in practicing the present invention, selection conditions may be chosen to minimize target-wall interactions, including the addition of wall modifications that minimize protein interactions (Horvath et al., Electrophoresis, 2001; 22:644-655; Righetti et al., Electrophoresis, 2001; 22:603-611). Some protein targets, especially membrane proteins, may not be compatible with purely aqueous selection conditions and will generally require the addition of surfactants or liposomes to prevent wall interactions.

Candidate Mixtures of Nucleic Acids:

The CE-SELEX selection procedure of the present invention is used to identify nucleic acid ligands of a target molecule from a candidate mixture of nucleic acids. Such a candidate mixture of nucleic acids may also be referred to as “a library,” “a combinatorial library,” “a random combinatorial library,” a “combinatorial pool,” a “random pool,” or a “randomized DNA pool.” Candidate mixtures of nucleic acids may be randomized pools of single stranded DNA or single stranded RNA. Libraries for use in CE-SELEX may be purchased commercially, for example, from Integrated DNA Technologies (Coralville, Iowa).

The libraries used for CE-SELEX may be similar to the randomized pools of DNA or RNA used in conventional SELEX (He et al., J. Mol. Biol., 1996; 255:55-66; Bock et al., Nature, 1992; 355:564-566). For example, a candidate mixture of nucleic acids, each nucleic acid sequence having a 40-base random region, flanked by two 20-base primers, similar to that used for IgE selections, may be used (see FIG. 4A). Or, for example, a candidate mixture of nucleic acids, each nucleic acid sequence having as a 75 base randomized region flanked by a 20-base primer and a 22-base primer, similar to that used for selection of ATP aptamers, may be used (see FIG. 4B).

Further, the primer sequences used in a library may be chosen to minimize primer-primer interactions or the formation of primer dimmers during PCR. Such primer sequences may also be chosen to optimize the melting temperature of the primers in PCR reactions. For example, the melting temperature of the primers may be altered. Primers may be selected with a melting temperature including, but not limited to, from about 45° C. to about 70° C. For example, a primer with a melting temperature of about 45° C., of about 46° C., of about 47° C., of about 48° C., of about 49° C., of about 50° C., of about 51° C., of about 52° C., of about 53° C., of about 54° C., of about 55° C., of about 56° C., of about 57° C., of about 58° C., of about 59° C., of about 60° C., of about 61° C., of about 62° C., of about 63° C., of about 64° C., of about 65° C., of about 66° C., of about 67° C., of about 68° C., of about 69° C., or of about 70° C. may be used in CE-SELEX. Primers may be selected with melting temperatures that are included in a range of any of the above melting listed temperatures. One example of a primer pair that may be used in CE-SELEX, with a melting temperature of about 59° C., are SEQ ID NO:123 and SEQ ID NO:124.

For example, in some CE-SELEX experiments, 100 nmol of DNA may be dissolved in a 50 μL aliquot of selection buffer. Based on an injection volume of 20 nL (typical when using a 50 mm diameter/50 cm long CE column) approximately 2.4×10¹³ DNA molecules are injected onto the CE column. This is smaller than the 10¹⁴-10¹⁵ sequences typically used in conventional SELEX, as a closer analysis reveals that libraries often do not need to be this large. For example, Wiegand et al. identified a 21 base conserved sequence in the IgE aptamer obtained using conventional SELEX (Wiegand et al., J. Immun., 1996; 157: 221-230). This motif could be placed in 20 positions on the 40 base randomized region used in the selection. The probability that an individual DNA molecule in the library would contain the binding sequence is 4.5×10⁻¹² (i.e. 0.2521×20). The probability that none of the molecules in a 10¹³ library contain the binding sequence is 1.8×10⁻²⁰ (i.e. (1-4.6×10⁻¹²). Table 2 lists the probability that different size libraries do not contain the binding sequence. A similar calculation can be performed for the ATP aptamer, which contains 22 conserved bases that can be placed in 49 positions within the 75 base random region (Battersby et al., J. Amer. Chem. Soc., 1999; 121:9781-9789). These calculations clearly show that the probability of a 2.4×10¹³ molecule library not containing a sequence that binds the target at least as well as those obtained using conventional SELEX is extremely small. It should also be noted that this does not account for mutagenesis during PCR, which will add diversity to the DNA pool during every round of selection.

TABLE 2
Probability that the binding sequence is not present in the initial DNA
library.

| Library Size | IgE Aptamer | ATP Aptamer |
|---|---|---|
| 10¹⁰ | 0.96 | 0.97 |
| 10¹¹ | 0.63 | 0.28 |
| 10¹² | 0.011 | 0.062 |
| 10¹³ | 1.8 × 10⁻²⁰ | 8.0 × 10⁻¹³ |
| 10¹⁴ | 3.2 × 10⁻¹⁹⁸ | 1.1 × 10⁻¹²¹ |
| 2.4 × 10¹³* | 2.0 × 10⁻⁴⁷ | 2.5 × 10⁻²⁹ |

*Library size in the proposed CE-SELEX experiments.

Aptamers:

The CE-SELEX process yields a class of products that are referred to as aptamers, each having a unique sequence. As used herein “aptamers” are nucleic acid ligands that have the property of binding specifically to a target compound or molecule. Thus, aptamers have a specific binding affinity for a three-dimensional target and exhibit molecular recognition. The resultant aptamers may be cloned and sequenced, allowing the production of large quantities of a single isolated and purified aptamer.

The CE-SELEX procedure of the present invention can be used to select aptamers that exhibit an affinity to a wide variety of targets. For example aptamers can be identified that bind to a large molecule target. Such large molecule targets may include, but are not limited to, IgE, Lrp, E. coli metJ protein, elastase, human immunodeficiency virus reverse transcriptase (HIV-RT), thrombin, T4 DNA polymerase, and L-selectin. Aptamers can also be identified with that bind to a small molecule target. Such small molecule targets may include, but are not limited to, ATP, L-arginine, kanamycin, lividomycin, neomycin, nicotinamide (NAD), N-methylmesoporphyrin (NMM), theophylline, tobramycin, D-tryptophan, L-valine, vitamin B12, D-serine, L-serine, γ-aminobutyric acid (γ-ABA), and organic dyes. Aptamers may also be identified that bind to macromolecules, including, but not limited to, viruses, such as human cytomegalovirus (HCMV), bacteria, eukaryotic cell, organelles, and nanoparticles.

The aptamers of the present invention will be of improved quality. Such aptamers will be useful as tools in analytical chemistry, useful in a wide range of diagnostic assays and will have direct benefits to many areas of research, including biomedical and health research. For example, increased binding efficiency and and/or increased binding selectivity will be beneficial in developing aptamer drugs that act on specific biological receptors. Aptamers with improved binding efficiency and selectivity will demonstrate increased pharmacological activity with fewer side effects. Improved aptamers will also be useful in developing diagnostic assays where detection limits are often related to binding affinity. Improved aptamers will also find use in many areas as diagnostic markers in, for example, medical analyses, in vivo imaging and biosensors. Improvements in selectivity will also be advantageous in quantitating targets present in complex matrices.

For example, aptamers of the present invention may be used to develop high-sensitivity affinity probe capillary electrophoresis (APCE) assays, similar to the IgE aptamer assay developed by German et al. (Anal. Chem., 1998; 70:4540-4545). Aptamers of the present invention may be used in ELISA type assays using enzyme-linked DNA aptamers, similar to the assay for bile acids developed by Kato et al., (Analyst, 2000; 125:1371-1373). Aptamers of the present invention may also be useful in imaging techniques, similar to the aptamer sequences that exhibit a fluorescence change in the presence of ATP developed by Jhaveri et al. (J. Amer. Chem. Soc., 2000; 122:2469-2473). Thrombin aptamers may be developed for use in fiber-optic microarray biosensors (see Lee et al., Anal. Biochem., 2000; 282:142-146). Aptamers against transformed endothelial cells may be selected for use as histological markers to identify tumor microvessels (see Blank et al., J. Biol. Chem., 20001; 279:16464-16468). Aptamers may be developed for use in other aptamer-based assays, such as assays for analytes ranging from anthrax spores (Bruno et al., Biosens. Bioelec., 1999; 14:457-464) to cocaine (Stojanovic et al., J. Am. Chem. Soc., 2001; 123:4928-4931).

The CE-SELEX procedure of the present invention may also be used to develop diagnostic assays for compounds of neurological interest—such as neuropeptides or small molecule neuromessengers, such as glutamate and zinc. It is often difficult to obtain high affinity antibodies against these neuromessengers, because they are widely abundant, making it difficult to induce an immune response. Aptamer based assays will not require such antibodies. Aptamer based diagnostic assays will also facilitate the analysis of neuropeptides, which are often present at picomolar concentrations in vivo. Thus, the present invention includes diagnostic assays using aptamers identified by the CE-SELEX procedure, including diagnostic assays for compounds of neurological interest.

The CE-SELEX approach for identifying molecules with an affinity for a target molecule will prove valuable in many areas of medical and biological research. For example, aptamers may be used as drugs, designed by selecting for molecules with affinity for certain biological receptors (see, for example, Osborne et al., Chem. Rev., 1997; 97:349-370; Brody et al., Rev. Mol. Biotech., 2000; 74:5-13; White et al., J. Clin. Invest., 2000; 106:929-934). Such aptamer drugs can be used to modify biological pathways or target pathogens, such as viruses or cancerous cells, for elimination. For example, aptamers that bind IgE inhibit immune response and may be useful in treating allergic reactions and asthma (Wiegand et al., J. Immun., 1996; 157: 221-230). Aptamers may be developed to bind thrombin, inhibit fibrin-clot formation, and may be useful in treating heart disease and preventing strokes (see, for example, Bock et al., Nature, 1992; 355:564-566; Dougan et al., Nuclear Med. Biol., 2000; 27:289-297). Aptamers may have antiviral applications. For example, aptamers have been selected with an RNA sequence that binds infectious human cytomegalovirus (HCMV) (Wang et al., RNA, 2000; 6:571-583). This HCMV aptamer prevents infection in vitro with a high specificity and may be useful in identifying viral proteins required for infectivity. Similarly, aptamers selected to bind HIV-1 RNase H exhibit antiviral activity in vitro (see Andreola et al., Biochem., 2001; 40:10087-10094).

The CE-SELEX procedure also opens the door to more fundamental studies involving biological molecules. Researchers have studied motifs (common sequences shared between active molecules) to gain a better understanding of the binding mechanisms of biological molecules (Famulok, J. Am. Chem. Soc., 1994; 116:1698-1706; (Conrad et al., Mol. Diversity, 1995; 1:69-78; Singer et al., Nucl. Acids Res., 1997; 25:781-786). Study of these motifs has also given insight into the process of evolution and why certain DNA and RNA sequences are conserved more rigidly between some species than others (Gold et al., Proc. Natl. Acad. Sci., 1997; 94:59-64; Shultzaberger et al., Nucl. Acids Res., 1999; 27:882-887; Landweber, Trends Ecol. Evol., 1999; 14:353-358).

The CE-SELEX method of the present invention may also be used in the selection of RNAs or DNAs that not only bind a target molecule, but also act as catalysts (see, Lorsch et al., Acc. Chem. Res., 1996; 29:103-110).

Aptamers of the present invention includes aptamers containing modified nucleotides conferring improved characteristics on the nucleic acid ligand, such as improved in vivo stability or improved delivery characteristics. Examples of such modifications include, but are not limited to, chemical substitutions at the ribose and/or phosphate and/or base positions. For example, such modified aptamers may contain nucleotide derivatives chemically modified at the 5- and 2′-positions of pyrimidines, as described in U.S. Pat. No. 5,660,985 or contain one or more nucleotides modified with 2′-amino (2′-NH₂), 2′-fluoro (2′-F), and/or 2′-O-methyl (2′-OMe), as described in U.S. Pat. No. 5,580,737.

Targets:

Molecules of any size or composition may serve as targets in the CE-SELEX method. For example, the target may be a protein, peptide, carbohydrate, polysaccharide, glycoprotein, hormone, receptor, antigen, antibody, virus, substrate, metabolite, transition state analog, cofactor, inhibitor, drug, dye, nutrient, growth factor, cell, or tissue.

The target may be a large molecule target. Such large molecule targets include, but are not limited to, IgE, Lrp, E. coli metJ protein, elastase, human immunodeficiency virus reverse transcriptase (HIV-RT), thrombin, T4 DNA polymerase, and L-selectin. The target may be a small molecule target. Such small molecule targets include, but are not limited to, ATP, L-arginine, kanamycin, lividomycin, neomycin, nicotinamide (NAD), N-methylmesoporphyrin (NMM), theophylline, tobramycin, D-tryptophan, L-valine, vitamin B12, D-serine, L-serine, γ-aminobutyric acid (γ-ABA), and organic dyes. As used herein, a “large molecule” is a molecule with a molecular weight greater than about 5 KDa. As used herein, a “small molecule” is a molecule with a molecular weight of about 5 KDa or smaller. In addition, aptamers can also be identified that bind to macromolecules, including, but not limited to, viruses, such as human cytomegalovirus (HCMV), bacteria, eukaryotic cell, organelles, and nanoparticles.

Polymerase Chain Reaction (PCR):

The volumes typically used in CE-SELEX are more compatible with PCR than those used in conventional SELEX. In conventional SELEX, the elution step of the chromatographic selection process results in the dilution of DNA concentrations that necessitates either a preconcentration step to minimize the volume or a larger scale PCR that consumes more reagents. The smaller volumes used in CE-SELEX, for example, aliquots of about 250 μL, are suitable for small scale PCR and are easily adjustable from 20-5000 μL.

Procedures for PCR methodologies are well known (see, for example, PCR Primer: A Laboratory Manual, (Cold Spring Harbor Laboratory Press, 1995)). PCR procedures for use in the CE-SELEX procedure may be performed using any of the various reaction conditions. For example, PCR may be performed using any of the various enzymes available, including, but not limited to, Taq polymerase and Taq polymerase in combination with a Taq-specific antibody (available as JUMPSTART Taq polymerase (Sigma-Aldrich, Corp., St. Louis, Mo.)). MgCl₂ concentrations in PCR procedures may be varied. For example, MgCl₂ concentrations may be varied from about 4.5 mM to about 10.5 mM MgCl₂; including, but not limited to, a MgCl₂ concentration of about 4.5 mM, about 6 mM, about 7.5 mM, about 9 mM, or about 10.5 mM. In PCR procedures, annealing temperatures may be varied. For example, annealing temperatures may range, from about 38° C. to about 60° C. Annealing temperatures may include, but are not limited to, 38° C., 40° C., 42° C., 44° C., 46° C., 48° C., 50° C., 52° C., 54° C., 55° C., 56° C., 58° C., or 60° C.

Primer sequences may also be chosen to optimize the melting temperature of the primers in PCR reactions. Primers may be selected with a melting temperature including, but not limited to, from about 45° C. to about 70° C. For example, a primer with a melting temperature of about 45° C., of about 46° C., of about 47° C., of about 48° C., of about 49° C., of about 50° C., of about 51° C., of about 52° C., of about 53° C., of about 54° C., of about 55° C., of about 56° C., of about 57° C., of about 58° C., of about 59° C., of about 60° C., of about 61° C., of about 62° C., of about 63° C., of about 64° C., of about 65° C., of about 66° C., of about 67° C., of about 68° C., of about 69° C., or of about 70° C. may be used in CE-SELEX. One example of a primer pair that may be used in CE-SELEX, with a melting temperature of about 59° C., are SEQ ID NO:123 and SEQ ID NO:124.

PCR may, for example, be performed using the Taq polymerase in a method similar to that used in the original SELEX selection for aptamers of IgE and ATP (Huizenga et al., Biochem., 1995; 34:656-665; Wiegand et al., J. Immun., 1996; 157: 221-230). Polymerase, primers, and nucleotides are added to the aliquot containing the active DNA. The 3′ primer is biotinylated giving a final PCR mixture where the complementary sequences are biotinylated and the original sequences are not, allowing for their separation before the next round of selection. Complementary sequences do not have affinity for the target and therefore need to be removed. The biotinylated complementary sequences are separated from the active sequences by passing the PCR mixture through a streptavidin-agarose column (Mitchell et al., Anal. Biochem., 1989; 178:239-242). The solution of ssDNA may then be used in an additional round of CE-SELEX selection. One or more rounds of selection may be required before most of the DNA shows affinity for the target.

Cloning and Sequencing:

The resultant ligand-enriched mixture obtained with the CE-SELEX procedure is still a mixture of aptamer sequences with similar binding affinities toward the target molecule. These differences may be minor (e.g. a similar sequence appearing at a different position on the aptamer) or may represent completely different binding mechanisms. Cloning and sequencing may be used to characterize individual aptamers, and to facilitate the identification of binding motifs. Any of the various cloning and sequencing procedures know to those of skill in the art may be used for the characterization of individual aptamers.

For example, twenty clones may be prepared using the TOPO TA Cloning Kit for Sequencing (Invitrogen). ssDNA sequences may be inserted into the pCR 4-TOPO vector using topoisomerase. The 3957 base pair vector may then be chemically transformed into Top 10 E. coli cells. After incubation, plasmid DNA may then be isolated using microcentrifuge extraction columns from the S.N.A.P. Miniprep Kit (Invitrogen). The restriction endonuclease EcoRI may then be used to remove the cloned sequence from the plasmid DNA for sequencing. The sequencing of individual clones may be performed by the University of Minnesota Advanced Genomic Analysis Center. The cloned sequences may be compared to identify similar binding motifs.

Binding Measurements:

The dissociation constants of the target-aptamer complexes of the present invention may be measured. For example, dissociation constants of the target-aptamer complexes of the present invention may be measured using ACE (Heegaard et al., J. Chrom. B, 1998; 715:29-54; Heegaard, Protein-Ligand Interactions: Hydrodynamics and Calorimetry, Harding et al., Eds., Oxford University Press, Oxford, 2001:171-195). The pre-incubation ACE procedure is often most successful with high affinity complexes. In this approach, a set concentration of target is incubated with varying concentrations of fluorescently labeled aptamer (0.1-100 times the expected K_{d}). CE is used to separate the aptamer bound to the target from the free aptamer. Laser induced fluorescence (LIF) detection is used to detect the low concentrations of the aptamer necessary to cover the optimum concentration range (i.e. 0.1-100 times the expected K_{d}). The concentration of the bound aptamer will vary according to:

$$[ complex ] = \frac{{[ target ]}_{tot} \times [ aptamer ]}{{K}_{d} + [ aptamer ]}$$

A plot of the aptamer-target complex concentration versus free aptamer concentration will take the shape of a rectangular hyperbola. A nonlinear regression will be used to estimate K_{d} directly from this curve. Monte Carlo simulations have demonstrated that nonlinear regression introduces less bias and error into the K_{d} estimate than linearized forms of equation 1 (Bowser et al., J. Phys. Chem. A, 1998; 102:8063-8071; Bowser et al., J. Phys. Chem. A, 1999; 103:197-202). If the equilibrium kinetics of any of the aptamer-target complexes are too fast to separate individual bound and free peaks, the dissociation constant will be measured using a mobility shift ACE assay (Heegaard et al., J. Chrom., 1998; B 715:29-54; Bowser et al., Electrophoresis, 1997; 18:82-91; Heegaard, Protein-Ligand Interactions: Hydrodynamics and Calorimetry, Harding et al., Eds., Oxford University Press, Oxford, 2001:171-195).

By definition, any aptamer obtained using CE-SELEX will have been selected to give a mobility shift upon binding to the target. No such guarantee can be made for aptamers selected using conventional SELEX. Thus the CE-SELEX procedure of the present invention will yield aptamers particularly suited for use in ACE-based diagnostic assays.

Kits:

The present invention also provides a kit for performing the CE-SELEX procedure. Such kits may include any of the components described herein necessary for performing the CE-SELEX procedure. For example, kits may include one or more of the following: capillary tubes suitable for CE, either coated or uncoated; a primer pair as described herein; a DNA or RNA combinatorial library; PCR reagents; CE separation buffer; streptavidin/agarose columns used in the preparation of single stranded DNA; transcriptase, for use with the screening of an RNA library; or reverse transcriptase, for use with the screening of a RNA library. Optionally, other reagents such as buffers and solutions needed to practice the invention may also be included. Kits of the present invention may be in a suitable packaging material in an amount sufficient for at least one procedure. Instructions for use of the packaged material are also typically included.

As used herein, the phrase “packaging material” refers to one or more physical structures used to house the contents of the kit. The packaging material is constructed by well-known methods, preferably to provide a sterile, contaminant-free environment. The packaging material has a label that indicates that the enclosed materials can be used for the CE-SELEX procedure. In addition, the packaging material may contain instructions indicating how the materials within the kit are to be employed to perform the CE-SELEX procedure. As used herein, the term “package” refers to a solid matrix or material such as glass, plastic, paper, foil, and the like. Thus, for example, a package can be a glass vial used to contain milligram quantities of a primer pair, a capillary tube filled with the appropriate running buffer, as described herein, or it can be a microtiter plate well in which a target molecule has been distributed. “Instructions for use” typically include a tangible expression describing the reagent concentration or at least one assay method parameter, such as the relative amounts of reagent and sample to be admixed, maintenance time periods for reagent/sample admixtures, temperature, buffer conditions, and the like. Instructions for use may include instructions for the modification of the instrument control and/and data collection software of a commercial CE instrument to facilitate fraction collection in the CE-SELEX procedure.

As used herein, an “isolated” molecule is a molecule, such as a nucleic acid ligand, that has been either removed from its natural environment, produced using recombinant techniques, or chemically or enzymatically synthesized. Preferably, a molecule, such as a nucleic acid ligand, is “purified,” i.e., essentially free from any other nucleic acids, polypeptides, or associated cellular products or other impurities.

Unless otherwise specified, “a,” “an,” “the,” and “at least one” are used interchangeably and mean one, or more than one.

### EXAMPLES

The present invention is illustrated by the following examples. It is to be understood that the particular examples, materials, amounts, and procedures are to be interpreted broadly in accordance with the scope and spirit of the invention as set forth herein.

### Example 1

A Comparison of the Effect of Kinetic Bias in SELEX and CE-SELEX

It is commonly assumed that SELEX provides aptamers with the optimum binding sequence. For example, many studies using SELEX to probe DNA-protein or RNA-protein interactions are based on this premise. As previously discussed, there are several sources of bias present in the SELEX process that could prevent the DNA/RNA pools from converging on the optimum binding sequence. Considering this potential for bias it would seem important to perform a control experiment to test if SELEX does truly provide the optimum binding sequence. Surprisingly, to our knowledge this has not been done.

A control experiment using a twenty base strand of ssDNA as a target will be performed. Using ssDNA as a target has the advantage of providing a priori knowledge of the optimum binding sequence (i.e. the complementary sequence to the target). DNA will also provide a target with high binding efficiency and selectivity for a particular sequence, making it the ideal choice for assessing the effectiveness of SELEX.

Both conventional SELEX and CE-SELEX will be assessed using the DNA target. The conditions of the conventional SELEX experiment will be similar to experiments described in the current literature (Battersby et al., J. Amer. Chem. Soc., 1999; 121:9781-9789; Wiegand et al., J. Immun., 1996; 157: 221-230). The DNA target will be synthesized (Integrated DNA Technologies) with a biotin moiety at the 5′ end. The target will then be loaded in excess onto a streptavidin-agarose column to prepare the affinity chromatography column. The initial DNA pool will contain 10¹⁵ sequences with a forty base random region flanked by two twenty base PCR primers. The DNA pool will be passed through a column containing streptavidin-agarose (no target) to remove any sequences that have non-specific affinity for the stationary support. The pool will then be loaded onto the affinity column. Sequences with no affinity will be washed off the column using selection buffer (50 mM TRIS, pH=7.2). Bound sequences will be eluted using five column volumes of elution buffer consisting of 50 mM TRIS, pH=7.2, and 1 pM of the target DNA. The target used to elute bound sequences will be modified at the 3′ end to prevent the possibility of extension taking place during PCR. Eluted sequences will be ethanol precipitated and redissolved in 50 mL of PCR buffer. The eluted DNA will then be amplified using PCR and made single stranded using the procedures described above. The single stranded PCR product will then be used in the next round of selection. After ten rounds of selection, twenty clones from the DNA pool will be sequenced to determine if conventional SELEX provided the correct complementary sequence to the DNA target. It is anticipated that kinetic bias will prevent SELEX from providing the correct complementary sequence. The slow dissociation kinetics of double stranded DNA will make elution of the optimum complementary sequence nearly impossible using the methods typically employed in SELEX. Instead, SELEX will probably provide partial complementary sequences that bind the target with intermediate affinity and can be eluted from the column.

For comparison, selection using the same DNA target will be performed using CE-SELEX. The initial DNA pool will be incubated with 1 pM of the target DNA. Selection will be performed as described above using a selection (CE) buffer containing 50 mM TRIS at pH=7.2. After ten rounds of selection, 20 clones from the DNA pool will be sequenced. It is anticipated that CE-SELEX will provide the correct complementary sequence to the DNA target since selection will take place in the solution phase, removing the potential for kinetic bias.

This will be the first time that a control experiment testing the assumption that SELEX provides the optimum binding sequence has been performed. This is an important experiment considering the increasing number of fundamental studies that are based on this assumption (Famulok, J. Am. Chem. Soc., 1994; 116:1698-1706; Conrad et al., Mol. Diversity, 1995; 1:69-78; Singer et al., Nucl. Acids Res., 1997; 25:781-786; Gold et al., Proc. Natl. Acad. Sci., 1997; 94:59-64; Shultzaberger et al., Nucl. Acids Res., 1999; 27:882-887; Landweber, Trends Ecol. Evol., 1999; 14:353-358). Demonstrating the presence of biases in conventional SELEX would also suggest that more attention should be given to optimizing selection procedures.

### Example 2

A Comparison of the Binding Affinity of Aptamers Selected Using SELEX and CE-SELEX

CE-SELEX selections will be performed against targets for which there are already well characterized aptamers. This will allow direct comparison of the binding affinity of the CE-SELEX and conventional SELEX aptamers. It is hypothesized that removing stationary phase biases using CE-SELEX will yield aptamers with improved binding efficiency.

Selections will be performed to identify aptamers with affinity for a large target (IgE) and a small target (ATP). Traditional SELEX typically performs better with large targets. This is usually attributed to the increased number of binding sites available for binding on large targets. Large targets will be somewhat easier with CE-SELEX as well since the DNA mobility shift will be larger than with smaller targets. IgE was chosen as the large target because it has previously been shown to be compatible with both CE and SELEX (German et al., Anal. Chem., 1998; 70:4540-4545). In addition the IgE aptamer selected using conventional SELEX has been well characterized (Wiegand et al., J. Immun., 1996; 157: 221-230), allowing comparisons of the binding sequences and affinities.

ATP will be used to test CE-SELEX with a small target. A significant concern is whether a small target will shift the DNA mobility enough to allow separation of the active sequences from the inactive ones. The high charge associated with ATP addresses this concern. Even though binding ATP will not have much affect on the size of the DNA, the addition of four negative charges will induce a mobility shift. This mobility shift has been confirmed in ACE experiments that used mobility shifts to measure ATP-aptamer binding (Battersby et al., J. Amer. Chem. Soc., 1999; 121:9781-9789). The conventional ATP aptamer has also been well characterized. The binding sequences have been identified (Huizenga et al., Biochem., 1995; 34:656-665), the three-dimensional structure of the binding pocket has been characterized using NMR (Lin et al., J. Chem. Biol., 1997; 4:817-832) and the affinity of the aptamers for ATP has been measured using ACE (Battersby et al., J. Amer. Chem. Soc., 1999; 121:9781-9789). These studies will allow direct comparisons between the ATP aptamer obtained using CE-SELEX and the conventional ATP aptamer.

The randomized DNA pools will be chosen to mimic the conventional SELEX experiments used to select the original IgE and ATP aptamers (Huizenga et al., Biochem., 1995; 34:656-665; Wiegand et al., J. Immun., 1996; 157: 221-230). A 40-base random region, flanked by two 20-base primers, will be used for the IgE selections (see FIG. 4A). The ATP selections will be performed on a DNA pool with a 75 base randomized region flanked by a 20-base primer and a 22-base primer (see FIG. 4B). The primer sequences were chosen to match those of the original experiments in case they affect the selection or binding.

CE selections will be performed as described above. The concentration of the target will be chosen such that it is high enough to ensure there are aptamers present that can bind the target at that concentration but low enough to select for the strongest binders. The dissociation constant of the IgE aptamer obtained using conventional SELEX is 10 nM (Wiegand et al., J. Immun., 1996; 157: 221-230), suggesting that a target concentration of approximately 40 nM would be appropriate to ensure a high fraction (80%) of the aptamers bind the target. The dissociation constant of the conventional ATP aptamer is 6 mM (Huizenga et al., Biochem., 1995; 34:656-665) suggesting a higher target concentration should be used for the ATP selections (e.g. 24 mM). It is anticipated that it will require 8-10 rounds of selection for the aptamer sequences to converge on the best binders. After selection, 20 clones will be prepared, allowing a direct comparison with the aptamers previously selected for IgE and ATP using conventional SELEX. Binding affinities will be measured using ACE as described above. The largest improvement in binding efficiency is expected with the ATP aptamer since the detrimental effect of the stationary phase linker is more pronounced with smaller targets.

These experiments will test the compatibility of electrophoretic separation with SELEX. The direct comparison with aptamers selected using CE-SELEX and conventional SELEX will determine if removing biases introduced by the stationary phase will yield aptamers with improved binding efficiency

### Example 3

A Comparison of the Binding Selectivity of Aptamers Selected Using SELEX and CE-SELEX

Although increased binding efficiency usually yields improved binding selectivity as well, this will not always be true. CE-SELEX should provide aptamers with improved selectivity because the DNA sequences will be able to interact with more sites on the target once the stationary phase linker has been removed.

N-methylmesoporphyrin (NMM, see FIG. 5) will be used as a target in the first selections testing the selectivity of aptamers obtained using CE-SELEX. This will allow direct comparison with aptamers selected using conventional SELEX. Li et al. used conventional SELEX to identify a 20-base DNA motif that binds NMM 4.5 times stronger than mesoporphyrin (Kd=0.8 and 3.6 mM, respectively) (Li et al., Biochem., 1996; 35:6911-6922). This was an impressive result considering the minimal structural difference between N-methylmesoporphyrin and mesoporphyrin (see FIG. 5A). Following the procedures for performing ACE experiments involving porphyrin acids (Bowser et al., Anal. Biochem., 1996; 241: 143-150; Bowser et al., Electrophoresis, 1997; 18:82-91), NMM/mesoporphyrin selection will be transferred to CE-SELEX.

After comparing CE-SELEX and conventional SELEX using NMM and mesoporphyrin as targets, two other sets of targets will be used to further test the selectivity of CE-SELEX. First an aptamer that preferentially binds D-serine over L-serine will be selected. This experiment will be interesting for two reasons. First it will test the ability of CE-SELEX to discover aptamers that demonstrate enantiomeric selectivity. D-serine is also of interest because it is thought to play a role in neurotransmission. D-serine has been implicated in numerous brain functions including learning, memory, stroke, epilepsy, Parkinson's, Alzheimer's disease and schizophrenia. Such an aptamer selective for D-serine could easily be incorporated into an affinity CE assay for D-serine.

The ability of aptamers selected using CE-SELEX to discriminate between structural isomers will also be tested. In these experiments we will select an aptamer that preferentially binds γ-aminobutyric acid (γ-ABA) over α-aminobutyric acid (α-ABA) and β-aminobutyric acid (β-ABA). γ-ABA is the major inhibitory neurotransmitter in mammals. Assays for γ-ABA are complicated by the presence of α-ABA and β-ABA. Therefore the identification of an aptamer selective for γ-ABA would not only be useful in assessing the ability of CE-SELEX generated aptamers to discriminate between structural isomers, but would also lead to the development of an affinity CE assay for this important neurotransmitter.

The randomized DNA pools will be prepared as discussed earlier. The specific sequences for the NMM/mesoporphyrin selection will be chosen to mimic the selection conditions of the original SELEX experiment (see FIG. 6). The primer sequences were chosen to match those of the original experiments in case they affect selection or binding. The D-serine/L-serine selection and the γ-ABA/α-ABA/β-ABA selection will be performed using the same sequences used to select the ATP aptamer. This will be the first time aptamers have been identified for these compounds so it is not necessary to mimic previous experiments.

CE selections will be performed as described above. A significant difference from the experiments described earlier will be introduction of negative selections into the procedure. In conventional SELEX, affinity columns can be used to eliminate sequences that bind compounds the researcher does not want the aptamer to show affinity for. We can perform a similar procedure in CE-SELEX. In the first round of selection, sequences that bind the target (e.g. NMM) will be collected and amplified as described above. In the second round of selection the separation buffer will be spiked with the compound that we want the aptamer to discriminate against (e.g. mesoporphyrin) instead of the target. In this round, sequences that do not exhibit a mobility shift (i.e. do not bind mesoporphyrin) will be passed to the next round of selection. We will alternate between positive selections for the target and negative selections against the compound we wish to discriminate against until the DNA sequence converges on an aptamer that selectively binds the target. Aptamers selected to bind NMM, D-serine and γ-ABA, as described earlier, will be cloned, sequenced and characterized. In particular the selectivity of the aptamers selected in these experiments will be assessed. The selectivity will be defined as the ratio of the dissociation constants for the aptamer/target and aptamer/non-target complexes. This selection will be performed directly against the actual target molecule in free solution, not bound to some stationary support. Thus, CE-SELEX will provide aptamers with improved selectivity over those obtained using conventional SELEX.

Aptamers offer tremendous potential as drugs or diagnostic agents. The successful application of aptamers in these fields hinges on developing methods for selecting aptamers with both high affinity and selectivity toward the target. It should be noted that high affinity does not ensure selectivity. The experiments described here will compare the selectivity of aptamers selected using CE-SELEX with those obtained using conventional SELEX. It is anticipated that the biases introduced by the stationary support limits both the affinity and selectivity of aptamers selected using conventional SELEX. Removing these biases in CE-SELEX should provide aptamers with improved affinity and selectivity, providing better drug and diagnostic targets.

### Example 4

Optimization of Selection Conditions

One advantage of CE-SELEX over conventional SELEX is the ease with which selection conditions can be modified. In conventional SELEX changing the number of binding sites in the selection column requires the preparation of an entirely new stationary phase. In CE-SELEX the capillary only needs to be rinsed with a selection buffer containing a different target concentration. This combined with the speed of the selection step in CE-SELEX (˜15-30 minutes) will allow experiments with different selection conditions to be run in parallel. The ease with which conditions can be modified and the ability to run multiple experiments in parallel will allow SELEX selection conditions to be studied with a level of detail not before possible.

One important selection variable in CE-SELEX is the target concentration. Some have suggested that the concentration of the target may define the binding affinity of the selected aptamers. During initial rounds of selection sequences that can bind the target are passed to the next round. If the target concentration remains constant it is not very long before most of the sequences can fully bind the target, removing any discrimination between sequences. There is no longer a driving force to evolve higher affinity aptamers. SELEX tends to produce aptamers that fully bind the target at the concentration used in selection, but not any stronger. The ease with which conditions can be changed in CE-SELEX will allow the target concentration to be gradually decreased on successive rounds of selection. This variation in target concentration will be tested using both IgE and ATP as targets. If decreasing the target concentration yields aptamers with improved binding the fixed target concentration typically used in conventional SELEX limits the selection process and should be abandoned.

Because of the difficulty of loading 10¹⁵ DNA molecules onto a capillary it will be important to determine what effect the size of the initial DNA library has on the binding efficiency of the resulting aptamers. We will perform selections with initial pool sizes ranging from 10¹²-10¹⁵ sequences. As discussed earlier, we expect to find that the initial library does not need to be as large as those typically used in conventional SELEX. It is anticipated that selections starting with smaller libraries may require a few extra rounds of selection but will still eventually converge on the optimum binding sequence.

In conventional SELEX negative selections are used to eliminate sequences that show affinity for the stationary support. Negative selection is performed by passing the DNA (or RNA) pool through a column containing underivatized stationary support. Sequences that bind the support are eliminated. Negative selection is potentially detrimental to the selection procedure because it eliminates sequences that can bind the stationary support even if they have strong affinity for the target. In CE-SELEX there are two effects that could cause a DNA sequence that does not bind the target to migrate in the collection window. A sequence that interacts with the capillary wall would migrate slower than the inactive DNA band. These interactions are expected to be rare since coated capillaries are designed to minimize DNA—wall binding. Another concern is that some sequences may have a particular secondary structure that causes them to nigrate either before or after the majority of the inactive sequences, even in the absence of the target. We will perform experiments to test if these sequences are abundant enough to require their removal through negative selection. Initial experiments will perform a negative selection after the first round. Negative selection will be performed by running a CE selection with no target in the selection buffer. DNA that migrate before or after the zone containing DNA with no affinity for the target will not be collected, eliminating sequences that migrate where the active DNA normally would, even in the absence of the target. Further experiments will perform negative selections between every round to determine the effect on the sequence and binding efficiency of the aptamers.

### Example 5

Determination of the Mobility Shift of the Template-Target Complex

A significant concern is whether CE will provide enough resolution to separate free DNA from sequences bound to a 20-base ssDNA target. In a SELEX experiment, the DNA sequences that bind the target have to be separated from the sequences that do not have affinity for the target. In this example, the mobility shift of the template DNA upon binding to the target DNA in the presence of the control DNA was determined. A 20-base ssDNA target (5′-CAT GGG CCA AGC TTC TTC GG-3′ (SEQ ID NO:118)), a 70-base ssDNA template (5′-CTA CCT ACG ATC TGA CTA GCT TTT TCC GAA GAA GCT TGG CCC ATG TTT TTG CTT ACT CTC ATG TAG TTC C-3′ (SEQ ID NO:119)), and a 70-base ssDNA control (5′-CTA CCT ACG ATC TGA CTA GCT TTT TTT TTT TTT TTT TTT TTT TTT TTT TTG CTT ACT CTC ATG TAG TTC C-3′ (SEQ ID NO:120)) were used to represent the target, sequences with affinity for the target, and sequences that do not have affinity for the target, respectively, for this mock CE-SELEX experiment. In a CE separation, the template-target complex will elute at a longer migration time compared to the unbound template. This experiment measures the resulting mobility shift of the template.

All samples and buffers were prepared using deionized water obtained from a Milli-Q water purification system (Millipore Corporation, Bedford, Mass.). The oligonucleotides used in this experiment were synthesized by Integrated DNA Technologies, Inc., Coralville, Iowa. The CE run buffer consisted of 20 mM Tris(hydroxymethyl)aminomethane (Tris) adjusted to a pH of 7.0. Prior to use, the CE buffer was filtered through a 0.2 μm membrane filter. All CE samples were prepared in a 10:90 buffer/water ratio.

A P/ACE MDQ Capillary Electrophoresis system (Beckman Coulter, Inc., Fullerton, Calif.) with 32 Karat software (Beckman Coulter, Inc., Fullerton, Calif.) was used to acquire the CE data. A 60.2 cm long by 50 μm inner diameter and 360 μm outer diameter uncoated fused-silica capillary (Polymicro Technologies, Phoenix, Ariz.) having a 50 cm length to detector was employed for the CE separations. At the beginning of each day the capillary was flushed with 0.1 M NaOH for 5 minutes and CE run buffer for 15 minutes. Before each CE run the capillary was rinsed with 0.1 M NaOH for 1 min and CE run buffer for 2 minutes. Samples were injected on to the capillary using the hydrodynamic mode and the samples were monitored using UV detection at 200 nm. The capillary cartridge was maintained at 25° C. and the sample chamber was held at 4° C. To drive the electrophoresis separation an electric field of 498 V/cm (0.17 min ramp) was applied.

The target is the 20-base ssDNA that will bind to a portion of the template sequence. The sequence of the template is similar to that typically used in a SELEX experiment. There is a 30-base “random” region flanked by two 20-base primer regions necessary for PCR amplification. In this case the correct complement to the target has been inserted into the “random” portion of the sequence. This sequence will bind the target strongly and is representative of the sequences that we will select in our actual CE-SELEX experiments. The control sequence is similar to that of the template except the “random” region has been replaced with poly(T). This sequence should show little affinity for the target and represented the inactive sequences in our mock selection.

FIG. 7A shows an electropherogram of a mixture of the template and control. As expected, these sequences are not separated in free solution. It should be noted that the separation efficiency is good, suggesting that DNA-capillary interactions are minimal. FIG. 7B demonstrates the separation obtained in the presence of the target DNA. Binding of the template to the target has caused an increase in the migration time of the template resulting in baseline resolution of the template from the control. Finally, FIG. 7C shows an electropherogram of a mixture containing only the template and the target demonstrating that even moderate concentrations of the target fully bind the template molecule.

To determine if PCR could be reliably performed on such a small DNA sample gel electrophoresis analysis was performed of PCR products from CE fraction collection. PCR conditions were 22 cycles (1 minute at 94° C., 1 minute at 41° C., 2.3 minutes at 72° C.), [dNTP]=1 mM, [primers]=1.5 μM, [Taq]=15 units, [MgCl₂]=7.5 mM. Electrophoresis conditions were 2% agarose, 30 minutes at 120 V. Gel electrophoresis analysis of the PCR products from this experiment clearly shows that PCR was successful in amplifying the DNA present in the collected fraction. A single band was detected at the correct position for a 70-base sequence of DNA. There was no evidence of smearing or tailing in the bands, suggesting that the PCR conditions were appropriate. And no band was observed in the no DNA control experiment, suggesting that false positives will not be a problem.

The next step in the SELEX process is to prepare the PCR products for subsequent rounds of selection. This involves removing excess PCR reagents, such as dNTP's, primers, and Taq polymerase. It is also necessary to make the double stranded PCR products single stranded before the next round of selection. In the current experiment, the complementary sequence would block interactions between the template and the target. In a selection against a non-DNA target the DNA will only take the correct three dimensional binding structure when single stranded.

The PCR products obtained from the CE fraction collection were purified using a phenol/chloroform extraction. The aqueous layer from this extraction was then passed through a streptavidin column. A biotinylated primer was used during PCR to generate the complementary sequences. The double stranded PCR products were therefore retained on the streptavidin column through the biotin linkage on the complementary strand. A NaOH rinse was used to denature the DNA, eluting the active sequences from the column. These sequences, now single stranded, were ethanol precipitated and redissolved in deionized water.

FIG. 8 shows an electropherogram of the amplified fraction after the clean-up procedure. The peak labeled with an asterisk is an unidentified contaminant from the PCR or the clean-up. CE buffer conditions were 20 mM Tris, 10 mM NaCl, pH 7.0 at 30 kV, 25° C., and UV detection at 200 nm. There is a peak at the correct migration time, suggesting that the clean-up procedure was successful in producing ssDNA. The efficiency of the peak is sufficient for subsequent rounds of selection. The concentration of ssDNA was estimated to be 1.9 nM. This concentration is most likely limited by the PCR amplification. PCR becomes less efficient at higher concentrations as the amplified DNA competes for the primer binding sites during the re-annealing step. This concentration will be sufficient for the subsequent rounds of selection since the number of active sequences in the DNA pool will be much more abundant after the first round of selection.

These results show that CE is able to provide enough separation efficiency to resolve the bound DNA sequences from the unbound sequences, indicating that collection of the bound DNA fraction, which will be used for PCR amplification, will be possible using this system.

### Example 6

Variation of Sample Ionic Strength for Optimum Binding

DNA binding interactions can vary greatly in sample solutions containing different amounts of salt (different ionic strengths). In general, the binding between complementary DNA strands becomes stronger in high ionic strength solutions. Moreover, the ionic strength of the buffer can have an impact on the separation efficiency in CE. In this experiment, CE run buffers containing different concentrations of NaCl were employed and the effects of ionic strength on the binding affinity and the CE separation efficiency of the target and template DNA were studied. In this experiment, CE buffers of different ionic strength were employed to test whether the binding between a DNA target and template could be improved.

The target sequence, the template sequence, the CE buffer, and the CE experimental details were the same as those used in Example 5.

FIG. 9A shows an electropherogram of a mixture of 2 μM template and 80 μM target in a run buffer that did not contain any NaCl. FIGS. 9A and 9C show electropherograms of a mixture of 2 μM template and 30 μM target in run buffers containing 10 and 20 mM NaCl, respectively. In FIG. 9D, a mixture of 2 μM template and 15 μM target in a run buffer containing 30 mM NaCl was used. The peaks for the complex and target elute at longer migration times at the higher NaCl concentrations because the electroosmotic flow in the capillary has decreased. This effect on the migration times results in a global shift of all the peaks in an electropherogram and is routinely observed in CE. The results in FIG. 9 indicate that as the ionic strength of the run buffer increases, a lower concentration of the target is needed to completely bind a 2 μM sample of the template. This clearly demonstrates that the binding between the target and the template gets stronger in the high ionic strength buffers. Additionally, FIG. 9 shows that there is a bigger migration time shift for the template-target complex in the high ionic strength buffers. It should be noted that run buffers containing 40 mM NaCl were also tried out for the separation of the template and target DNA. However, this high concentration of NaCl led to very high currents in the CE instrument. High currents in CE result in increased joule heating inside the capillary, which causes deterioration in the separation efficiency of CE.

These results show that by increasing the ionic strength of the buffer, better binding of the target to the template, and improved resolution of the bound and unbound DNA can be achieved in a CE-SELEX experiment. The enhanced binding will allow the use of lower concentrations of the target in CE-SELEX. The improved resolution of the bound from the unbound DNA will allow a greater proportion of the inactive DNA sequences to be eliminated in each CE-SELEX round and will shorten the CE-SELEX experiment considerably as fewer rounds would be required to obtain the final pool of active DNA. These improvements need to be balanced against increased current and Joule heating that occurs as the ionic strength increases. In this experiment, 30 mM NaCl was determined to provide the optimum ionic strength for the current separation conditions.

### Example 7

Mock CE-SELEX Enrichment of Template DNA in Excess of Control DNA

In this experiment, the selectivity of CE-SELEX towards a small amount of template DNA and an excess of the control DNA was addressed. A mixture of the template (representing active sequences) and control (representing inactive sequences) was used. Using the 20-base target DNA (SEQ ID NO:118), several CE-SELEX rounds were performed in order to enrich the template while eliminating as much of the control as possible.

The target sequence, the template sequence, the control sequence, and the CE experimental details were the same as those in Example 5. The CE run buffer consisted of 20 mM Tris and 20 mM NaCl at a pH of 7.0. The CE fractions containing the active DNA were performed as follows: After injecting the CE samples, electrophoresis was allowed to proceed until the inactive sequences eluted off the capillary. Then the voltage was switched off and the active sequences, which migrate at a slower rate compared to the inactive sequences, were removed from the capillary by applying a pressure rinse using the buffer.

The DNA sequences in the CE fraction were amplified using the polymerase chain reaction (PCR). The PCR amplifications were performed with two 20-base ssDNA primers, primer 1 (5′-CTA CCT ACG ATC TGA CTA GC-3′) (SEQ ID NO:121) and primer 2 (5′-/Biotin/GGA ACT ACA TGA GAG TAA GC-3′) (SEQ ID NO:122). The PCR reagent mix consisted of 1 mM deoxyribonucleotide triphosphates (dNTP), 1.5 μM of primer 1, 1.5 μM of primer 2, 15 units of Taq enzyme, and 7.5 mM MgCl2. The PCR cycling conditions were begun with an initial denaturation at 94° C. followed by 22 cycles, which included denaturation for 1 minute at 94° C., annealing for 1 minute at 41° C., and extension for 2.3 minutes at 72° C. After the 22 cycles were complete, a final extension was carried out for 10 minutes at 72° C. In all cases a control PCR amplification was performed with all the PCR reagents listed above but without any added DNA. Success of the PCR reaction was verified by running aliquots of the PCR samples and control on a 2% agarose gel stained with ethidium bromide.

Following PCR, the DNA was phenol-chloroform extracted to remove unreacted Taq polymerase. The DNA in the aqueous layer of the phenol-chloroform extract was then made single stranded by passing it through a streptavidin-agarose (Pierce Biotechnology, Rockford, Ill.) column. Prior to use the streptavidin-agarose on the column was washed and equilibrated with binding buffer (10 mM Tris, 50 mM NaCl, and 1 mM EDTA at pH 7.5) for 5 minutes. The phenol-chloroform extract was added to the equilibrated streptavidin-agarose and was held for 30 minutes with occasional shaking. The double stranded PCR products were retained on the streptavidin column through the biotin linkage on the complementary strand because a biotinylated primer was used during PCR to generate the complementary sequences. The column was then washed 10 times with 1 mL binding buffer. Then 300 μL of 0.5 M NaOH was added to the streptavidin-agarose and the mixture was removed from the column and incubated at 37° C. for 15 minutes. The suspension was returned to the column and the ssDNA solution was eluted off the column and collected. The process was repeated with another 300 μL aliquot of 0.5 M NaOH. The NaOH rinse was used to denature the DNA, eluting the active sequences from the column while retaining the biotinylated complementary strand on the column.

Ethanol precipitation was used to recover the DNA from the two ssDNA solutions obtained from the end of the streptavidin-agarose step. To each 300 μL ssDNA solution, 1 mL of ice-cold ethanol and 30 μL of 3M sodium acetate (pH 4.5) was added. The solutions were mixed and stored at −80° C. for 1 hour. The solutions were then centrifuged for 20 minutes at 4° C. and 14,000 rpm. The supernatant solution was then decanted and discarded. The resulting DNA pellet was washed once with 1 mL of ice-cold 70:30 ethanol/water and centrifuged for 10 minutes at 4° C. and 14,000 rpm. The DNA pellets were then dried in a vacuum drier and dissolved in 20 μL of water. This DNA solution was used in subsequent CE-SELEX rounds.

FIG. 10 is an electropherogram of the initial CE-SELEX cycle in which 100 μM control, 2 μM template, and 30 μM target was used. The CE fraction containing the bound template was collected, PCR amplified, made single stranded, and then used in the subsequent CE-SELEX cycle. After only two CE-SELEX cycles, a significant enrichment of the template over the control was obtained. FIG. 11 shows electropherograms that demonstrate these results. FIG. 11A is an electropherogram of 10 μL of the single stranded DNA from the end of the second CE-SELEX cycle. When the sample from FIG. 11A was spiked with 20 mM target, the large peak from FIG. 11A shifted to a longer migration time (FIG. 11B), which was consistent with the expected migration time for the template-target complex. Note that most of the DNA in FIG. 11B exhibits a mobility shift, suggesting binding with the target. This suggests that the large peak in FIG. 11A was indeed the enriched target ssDNA. The amount of template used in the initial CE-SELEX cycle was 2%. Using the peak heights for the template and control peaks in the electropherograms obtained at the end of the first, second, and third cycles the amounts of the template were 65.6, 81.4, and 74.5%, respectively. Thus, we have shown that CE-SELEX is able to select the desired DNA sequence and enrich it while removing the undesired sequences from the DNA pool. Since the enrichment factor of the template tended towards a plateau after the third cycle, additional CE-SELEX cycles were not performed.

Next, we performed the actual CE-SELEX experiment beginning with a combinatorial library of 70-base long ssDNA, which consisted of a 30-base “random” region flanked by two 20-base primer regions. The target DNA sequence used was the same as the one used earlier in the mock CE-SELEX experiments. The goal of this experiment was to test whether CE-SELEX was able to select the correct complementary sequence to the target from the library of randomized DNA sequences. Initially, a 2 mM sample of the library was injected onto the capillary. An injection of this size corresponds to approximately 2.4×10¹³ different DNA sequences. A large library is required to ensure that the probability of the correct DNA sequence being present in the initial library is high. However, we found that injecting such a high concentration of the library yielded poor results in the CE-SELEX experiment. Four CE-SELEX cycles were performed and in all cases the single stranded DNA obtained yielded a broad peak in the electropherograms, which indicated that little or no enrichment was occurring. Thus, we decided to inject a 1 mM sample of the library in the initial cycle.

FIG. 12 shows an electropherogram of the initial CE-SELEX cycle consisting of a sample of 1 μM library and 5 μM target. CE conditions were 20 mM Tris, 20 mM NaCl, pH=7.0 buffer, 30 kV, 25° C., and UV detection at 200 nm. As shown in FIG. 13, after two CE-SELEX cycles, a better-defined peak of single stranded DNA was observed. A CE fraction was collected in the expected migration window of the target-template complex and was then used in a subsequent CE-SELEX cycle. CE conditions were 20 mM Tris, 20 mM NaCl, pH=7.0 buffer, 30 kV, 25° C., and UV detection at 200 nm. Unfortunately, the gel electrophoresis analysis of the PCR products from this fraction did not produce a DNA band in the position for a 70-base sequence. Most likely, this suggests that too little or none of the correct sequence was present at the end of the second CE-SELEX cycle (FIG. 13). This, in turn, could be caused by not using a large enough library. In future CE experiments, we will use capillaries with larger inner diameters than the ones used in the experiments discussed here. In this way, one is able to work with smaller concentrations of the initial library while still injecting a sufficiently large number of different DNA sequences.

This experiment has demonstrated all of the procedural steps of the CE-SELEX process. Separation of binding and non-binding sequences in the presence of a 20-base DNA target was sufficient for fraction collection. PCR amplification of the collected sequences was also demonstrated. Procedures for preparing the PCR products for subsequent rounds of selection were shown to be successful. Moreover, CE separation of the product of these procedures gave a peak corresponding to ssDNA suitable for further selection. Significantly, with only two cycles of CE-SELEX, 81.4% enrichment was achieved for the template over the control. This represents a much higher enrichment rate than would be expected for conventional SELEX.

### Example 8

Prevention of Sample Destacking in CE After Each CE-SELEX Round

After making the active DNA single stranded and concentrating it by ethanol precipitation, it was noticed that for several of the samples the CE peak shape was somewhat broad. This was most likely being caused by destacking of the sample zone after injection. In CE, if the sample solution injected on to the capillary has a higher ionic strength than the run buffer, destacking or a dispersion of the sample band usually occurs. This suggested that the ionic strength of the ssDNA solutions at the end of each CE-SELEX round was relatively high for proper analysis. In this Example, to improve the CE peak shape of the ssDNA for subsequent CE-SELEX rounds, the step in which the active DNA is made single stranded was modified to reduce the ionic strength of the final ssDNA solution.

The target sequence, the template sequence, and the CE experimental details were the same as those in Example 5. The CE run buffer, the CE fraction collection, the PCR amplification, the procedure for generating ssDNA (except for the modifications listed below in the Results section), and the ethanol precipitation step were the same as in Example 7.

A CE-SELEX round was initiated with 2 μM of the template DNA, 80 μM of the control DNA, and 80 μM of the target DNA. The CE fraction containing the active DNA was collected, PCR amplified and made single stranded. FIG. 14 is an electropherogram of the ssDNA from the end of this initial CE-SELEX round spiked with 80 μM of the target DNA. It is clear from FIG. 14 that the CE sample band is significantly dispersed. In fact, the sample zone is so distorted that no separation of the ssDNA and target DNA occurs. This would make collection of an additional CE fraction for the next CE-SELEX round extremely difficult, if not impossible. This sample distortion is caused by the high ionic strength of the sample solution. In order to decrease the ionic strength of the sample, lower concentrations of NaOH were used to denature the complementary biotinylated strand in the streptavidin-agarose column. For the CE-SELEX cycle shown in FIG. 14, two 300 μL aliquots of 0.5 M NaOH were added to the streptavidin-agarose column to denature the biotinylated strand. To decrease the ionic strength of the final ssDNA solution, the amount and concentration of NaOH was reduced to two 200 μL aliquots at a concentration of 0.15 M. FIG. 15 shows an electropherogram of the ssDNA from the end of one CE-SELEX round performed with a reduced amount of NaOH plus 30 μM target DNA. Two distinct peaks for the ssDNA (earlier peak) and target DNA (latter peak) can now be seen. This improved resolution and peak shape will now make collection of the active sequences possible allowing additional CE-SELEX rounds to be performed.

Using both a smaller amount and a lower concentration of NaOH to denature the DNA while making it single stranded reduces the ionic strength of the final ssDNA solution. This prevents sample destacking in CE from occurring and results in improved CE peak shapes for subsequent CE-SELEX rounds.

### Example 9

Optimization of PCR Amplification of DNA

The protocol for PCR amplification of DNA in CE fractions that was used in Examples 7 and 8 was found to be unreliable when very low amounts of starting DNA was used. In most samples primer-dimers that were about 40 bases long would form in addition to the desired DNA, which was 70 bases long. Also, in many cases the PCR amplification from high DNA copy numbers did not result in successful amplification. In this example, the results of experiments to optimize the PCR amplification of DNA in the CE-SELEX process for the amplification of low amounts of DNA are presented.

The template DNA and the control DNA sequences were the same as were used in Example 5. The PCR conditions were the same as were outlined in Example 7, except for the changes noted below in the results section.

Variation of MgCl₂ concentration: The MgCl₂ concentration in a PCR reaction can be important for proper amplification of the template DNA. In this experiment, the concentration of MgCl₂ in the PCR reaction mixture was varied from 4.5 to 10.5 mM. FIG. 16 shows a photograph of a 2% agarose gel stained with ethidium bromide containing PCR samples run at different MgCl2 concentrations. PCR Conditions were 20 cycles (1 minute at 94° C., 1 minute at 42° C., 2.3 minutes at 72° C.), 1 mM dNTP, 1.5 μM primers, 15 units Taq. Electrophoresis conditions were 2% agarose, 30 minutes at 120 V. The lane assignments in FIG. 16 were as follows: lane 1—1,000 starting DNA molecules and 4.5 mM MgCl₂; lane 2—1,000,000 starting DNA molecules and 4.5 mM MgCl₂; lane 3—Control PCR with 4.5 mM MgCl₂; lane 4—1,000,000 starting DNA molecules and 6.0 MM MgCl₂; lane 5—Control PCR with 6.0 mM MgCl₂; lane 6—1,000 starting DNA molecules and 7.5 mM MgCl₂; lane 7—1,000,000 starting DNA molecules and 7.5 mM MgCl₂; lane 8—Control PCR with 7.5 mM MgCl₂; lane 9—25 bp DNA step ladder; lane 10—100 bp DNA step ladder; lane 11—1,000,000 starting DNA molecules and 9.0 mM MgCl₂; lane 12—Control PCR with 9.0 mM MgCl₂; lane 13—1,000 starting DNA molecules and 10.5 mM MgCl₂; lane 14—1,000,000 starting DNA molecules and 10.5 mM MgCl₂; lane 15—Control PCR with 10.5 mM MgCl₂. In all PCR sample and control reactions only a primer-dimer band about 40 bases long was seen, suggesting that the some factor other than the MgCl₂ concentration is responsible for the lack of PCR amplification success.

Variation of annealing temperatures: The annealing temperatures used in each PCR cycle can also have an effect on the quality of the PCR reaction. Higher annealing temperatures usually result in more specific amplification but the product yield is less compared to lower annealing temperatures. In this study the different annealing temperatures used were 38, 42, 44, 46, 50, 52, 55, and 60° C. Moreover, for the 44, 46, 50, 52, and 60° C. annealing temperatures the MgCl₂ concentration was also varied. FIG. 17 shows a photograph of a 2% agarose gel stained with ethidium bromide containing PCR samples that were run at annealing temperature of 46° C. and different MgCl₂ concentrations. PCR conditions were 20 cycles (1 minute at 94° C., 1 minutes at 46° C., 2.3 minutes at 72° C.), 1 mM dNTP, 1.5 μM primers, 15 units Taq. Electrophoresis conditions were 2% agarose and 30 minutes at 120 V. The lane assignments in FIG. 17 were as follows: lane 1—1,000 starting DNA molecules and 4.5 mM MgCl₂; lane 2—1,000,000 starting DNA molecules and 4.5 mM MgCl₂; lane 3—Control PCR with 4.5 mM MgCl₂; lane 4—1,000,000 starting DNA molecules and 6.0 mM MgCl₂; lane 5—Control PCR with 6.0 mM MgCl₂; lane 6—1,000 starting DNA molecules and 7.5 mM MgCl₂; lane 7—1,000,000 starting DNA molecules and 7.5 mM MgCl₂; lane 8—Control PCR with 7.5 mM MgCl₂; lane 9—25 bp DNA step ladder; lane 10—empty lane; lane 11—1,000,000 starting DNA molecules and 9.0 mM MgCl₂; lane 12—Control PCR with 9.0 mM MgCl₂; lane 13—1,000 starting DNA molecules and 10.5 mM MgCl₂; lane 14—1,000,000 starting DNA molecules and 10.5 mM MgCl₂; lane 15—Control PCR with 10.5 mM MgCl₂. For all samples shown in FIG. 17, primer-dimer bands about 40 bases long are detected and no template DNA bands are detected, indicating that the PCR reactions failed. Also, none of the other annealing temperatures used in this study resulted in successful PCR amplification of the template DNA.

Hot-start PCR: In a regular PCR reaction, all reagents are mixed at room temperature and then thermal cycling is initiated at the desired temperature. However, at room temperature the PCR primers can interact with each other and Taq polymerase is then able to extend these non-specific sequences. Since there are only a few of the template DNA sequences present at the beginning of a PCR reaction, even a small number of non-specific sequences can effectively compete with the template DNA for primer binding and end up being amplified. The most common way to prevent this is to perform a hot-start PCR. The simplest way to do a hot-start PCR is to add the Taq enzyme while the PCR sample is at 94° C., not at room temperature. Another, more reliable way to do a hot-start PCR is to use Taq polymerase supplied with an antibody that binds to Taq at room temperature rendering Taq inactive. When the solution is heated to about 70° C., the antibody is destroyed and Taq functions as it normally would. Both methods prevent extension of non-specific sequences. In our work, we have employed both the manual hot-start method and have used Taq supplied with an antibody (JUMPSTART Taq DNA Polymerase, Sigma-Aldrich Corp., St. Louis, Mo.). However, for both hot-start methods we were not able to observe a band for the template DNA. Instead bands at about 40 bases long were seen which are primer-dimers. Thus, the hot-start method was also not successful at amplifying the template DNA.

PCR with new primers: Having tried several optimization strategies for low copy-number PCR without success, it seemed possible that the primers were the source of the problem. The PCR primers used thus far were the same as the ones used in a conventional SELEX selection for immunoglobulinE (IgE). Primer 1 had a melting temperature of 52° C. and primer 2 had a melting temperature of 51 ° C. When choosing primers for PCR, primers with high melting temperatures are more likely to be successful because they will be able to prime to the template DNA at a temperature that is high enough to prevent mispriming and other non-specific interactions. Using this fact and other criteria commonly used for the selection of primers in PCR, we designed a new primer pair, primer 1 (5′-AGC AGC ACA GAG GTC AGA TG-3′ (SEQ ID NO:123)) and primer 2 (5′-/Biotin/TTC ACG GTA GCA CGC ATA GG-3′ (SEQ ID NO:124)). The new primer 1 and primer 2 both had a melting temperature of 59° C., allowing higher annealing temperatures to be used in the PCR reactions. Since new primers were being used, the primer regions on the template DNA sequence had to be changed as well. The sequence of the new template DNA was 5′-AGC AGC ACA GAG GTC AGA TGT TTT TCC GAA GAA GCT TGG CCC ATG TTT TTC CTA TGC GTG CTA CCG TGA A-3′ (SEQ ID NO:125). FIG. 18 is a photograph of a 2% agarose gel stained with ethidium bromide containing PCR samples obtained by amplifying a CE fraction containing the template DNA with the new PCR primers. PCR conditions were 24 cycles (30 seconds at 94° C., 30 seconds at 53° C., 20 seconds at 72° C.), 1 mM dNTP, 1.5 μM primers, 15 units Taq, and 7.5 mM MgCl₂. Electrophoresis conditions were 2% agarose, 30 minutes at 120 V. Lanes 1 to 3, 5 to 7, 9, and 10 are PCR samples containing the template DNA. Lanes 4 and 8 contain the 25 bp DNA step ladder, and lane 11 contains the PCR control reaction. Note, that in this PCR experiment, different cycling times were used. The results in FIG. 18 show that for all eight PCR samples, a band in the correct position on the gel (70 bases long) is observed and no other bands are seen. The control experiment has a band about 100 bases long. This is most likely the result of PCR primers interacting with themselves and being extended. This could happen because the control PCR did not contain any template DNA and the primers had nothing else to prime to. Importantly, there are no bands that are 100 bases long for the sample lanes. Thus, these results show that PCR amplification with the new primers works well and is able to successfully amplify the template DNA. Also, PCR reactions with the new primers using different annealing temperatures (52 and 54° C.) were tried out and they were also successful in amplifying the template DNA. The product yield for the 70 base template DNA was essentially the same for all three annealing temperatures studied. Thus, 53° C. was chosen as the annealing temperature for PCR reactions with these primers.

Several attempts at making the PCR reaction work with the old primers were not successful. However, with completely redesigned primers, which had higher melting temperatures, the PCR reaction worked extremely well. All PCR reactions from this point on were performed with this new primer pair.

### Example 10

CE-SELEX Selections Against ImmunoglobulinE (IgE)

To test the effectiveness of the CE-SELEX method and to compare the CE-SELEX DNA aptamers with those obtained from a conventional SELEX selection, aptamers were selected from a DNA library using IgE as a target. Immunoglobulins (Ig) are glycoproteins that function as antibodies. They are produced by plasma cells in response to an immunogen. Of all the Ig present in serum, IgE is the least common and takes part in allergic reactions. IgE binds very tightly to basophils and mast cells. When allergens bind to IgE on the cells, several pharmacological mediators, including histamine, are released and these mediators give rise to allergic symptoms observed in patients. DNA aptamers that bind strongly to IgE can inhibit binding of IgE to cells and allergens, thus preventing allergic symptoms. Thus, IgE aptamers will be useful pharmaceutical agents for the treatment of allergies and other IgE-mediated conditions. IgE aptamers with good binding efficiencies have been selected using conventional SELEX. Thus, using IgE as a target in CE-SELEX selections will allow the effective comparison of the CE-SELEX technique to conventional SELEX.

The DNA library used in this experiment was synthesized by Integrated DNA Technologies Inc. (Coralville, Iowa) and consisted of a 40 base random region of the four natural bases (G, C, T, and A) in a 25:25:25:25 ratio (5′-AGC AGC ACA GAG GTC AGA TG-(40 random bases)-CCT ATG CGT GCT ACC GTG AA-3′ (SEQ ID NO:126)). The 40 base random region was flanked by two 20 base primer regions, which matched the primer sequences that were designed in Example 5. IgE (human plasma, monoclonal, kappa light chain) was obtained from Athens Research and Technologies, Athens, Ga. The CE run buffer was a PBS buffer containing MgCl₂ and consisted of 8.1 mM Na₂HPO₄, 1.1 mM KH₂PO₄, 1 mM MgCl₂, 2.7 mM KCl, and NaCl (35 mM NaCl for the first CE-SELEX selection and 40 mM NaCl for the second and third CE-SELEX selections) at a pH of 8.0. Prior to use, the CE buffer was filtered through a 0.2 μm membrane filter.

A P/ACE MDQ Capillary Electrophoresis system (Beckman Coulter, Inc., Fullerton, Calif.) with 32 Karat software (Beckman Coulter, Inc., Fullerton, Calif.) was used to acquire the CE data. A 50.2 cm long by 50 μm inner diameter and 360 μm outer diameter polyacrylamide coated capillary (Beckman eCAP neutral capillary, Beckman Coulter, Inc., Fullerton, Calif.) having a 40 cm length to detector was employed in the first CE-SELEX selection. The second CE-SELEX selection was performed with a Beckman eCAP N—CHO coated capillary (Beckman Coulter, Inc., Fullerton, Calif.) with the same dimensions as the capillary used in the first selection. The third CE-SELEX selection was performed with a 50 μm inner diameter by 360 μm outer diameter Beckman eCAP N—CHO coated capillary, but with a total length of 40.2 cm and a length to detector of 30 cm. At the beginning of each day the capillaries were flushed with CE run buffer for 15 minutes. Before each CE run the capillary was rinsed with CE run buffer for 2 minutes. Samples were injected on to the capillary using the hydrodynamic mode and the samples were monitored using UV detection at 254 nm. The capillary cartridge was maintained at 25° C. and the sample chamber was held at 4° C. To drive the electrophoresis separations electric fields of 598 V/cm (first and second selection) and 498 V/cm (third selection) were utilized.

The active DNA sequences in each CE fraction were amplified using a manual hot-start PCR reaction. The PCR amplifications were performed with two 20-base ssDNA primers, primer 1 (5′-AGC AGC ACA GAG GTC AGA TG-3′ (SEQ ID NO:123)) and primer 2 (5′-/Biotin/TTC ACG GTA GCA CGC ATA GG-3′ (SEQ ID NO:124)). The PCR reagent mix consisted of 1 mM deoxyribonucleotide triphosphates (dNTP), 1.5 μM of primer 1, 1.5 μM of primer 2, 15 units of Taq enzyme, and 7.5 mM MgCl₂. The PCR cycling conditions were begun with an initial denaturation at 94° C. for 5 minutes (Taq enzyme was added to the reaction mixture at this stage) followed by 20 cycles, which included denaturation for 30 seconds at 94° C., annealing for 30 seconds at 53° C., and extension for 20 seconds at 72° C. After the 20 cycles were complete, a final extension was carried out for 5 minutes at 72° C. In all cases a control PCR amplification was performed with all the PCR reagents listed above but without any added DNA. Success of the PCR reaction was verified by running aliquots of the PCR samples and control on a 2% agarose gel stained with ethidium bromide.

Following PCR, the DNA was made single stranded by passing it through a streptavidin-agarose column (Pierce Biotechnology, Rockford, Ill.). Prior to use the streptavidin-agarose on the column was washed and equilibrated with binding buffer (10 mM Tris, 50 mM NaCl, and 1 mM EDTA at pH 7.5) for 5 minutes. The DNA from the PCR reaction was added to the equilibrated streptavidin-agarose and was held for 30 minutes with occasional shaking. The column was then washed 10 times with 1 mL binding buffer. Then 200 μL of 0.15 M NaOH was added to the streptavidin-agarose and the mixture was removed from the column and incubated at 37° C. for 15 minutes. The suspension was returned to the column and the ssDNA solution was eluted off the column and collected. The process was repeated with another 200 μL aliquot of 0.15 M NaOH.

Ethanol precipitation was used to recover the DNA in the two ssDNA solutions obtained from the end of the streptavidin-agarose step. To each 200 μL ssDNA solution, 200 μL of 0.15 M acetic acid, 1 mL of ice-cold ethanol, and 30 μL of 3 M sodium acetate (pH 4.5) were added. The solutions were mixed and stored at −80° C. for 1 hour. The solutions were then centrifuged for 20 minutes at 4° C. and 14,000 rpm. The supernatant solution was then decanted and discarded. The resulting DNA pellet was washed once with 1 mL of ice-cold 70:30 ethanol/water and centrifuged for 10 minutes at 4° C. and 14,000 rpm. The DNA pellets were then dried in a vacuum drier and dissolved in 15 μL of the CE buffer.

DNA clones were obtained using the ssDNA from the end of the second round of selection in the first and second CE-SELEX experiments, and from the end of the second and fourth rounds in the third CE-SELEX experiment. A small portion of the ssDNA solution was PCR amplified using primer 1 (5′-AGC AGC ACA GAG GTC AGA TG-3′ (SEQ ID NO:123)) and a non-biotinylated version of primer 2 (5′-TTC ACG GTA GCA CGC ATA GG-3′ (SEQ ID NO:124)). The PCR reagent mix consisted of 1 mM deoxyribonucleotide triphosphates (dNTP), 1.5 μM of primer 1, 1.5 μM of primer 2, 15 units of Taq enzyme, and 7.5 mM MgCl₂. The PCR cycling conditions were begun with an initial denaturation at 94° C. for 5 minutes (Taq enzyme was added to the reaction mixture at this stage) followed by 8 cycles, which included denaturation for 30 seconds at 94° C., annealing for 30 seconds at 53° C., and extension for 20 seconds at 72° C. After the 8 cycles were complete, a final extension was carried out for 10 minutes at 72° C. For each sample ˜30 clones were obtained. Cloning and sequencing of the DNA from the end of the PCR reactions was performed at the BioTechnology Resource Center at the University of Minnesota, St. Paul, Minn.

Dissociation constants (K_{d}) for representative clones from the CE-SELEX experiments were obtained using affinity capillary electrophoresis (ACE). The capillary and CE run buffer used were the same as were used for the CE-SELEX selections. Detection in the CE instrument was done using laser-induced fluorescence (LIF). Excitation was achieved with the 488 nm line of an Ar+ laser and the emitted fluorescence was collected at 520 nm. Samples containing 15 nM of the DNA were incubated with different amounts of IgE and then injected on to the capillary. The peak heights of the unbound DNA were used for calculating the K_{d} values. A nonlinear least-squares regression analysis using GraphPad Prism version 4.00 (GraphPad Software, San Diego, Calif. USA) was used to determine the K_{d} values.

First CE-SELEX selection against IgE: In this experiment, the first CE-SELEX round was performed with a sample solution containing 1.6 mM of the 80-base DNA library, 1 mM MgCl₂, and 1 μM IgE. Prior to injecting the sample on the CE column, it was allowed to sit at room temperature for 30 minutes. After the inactive DNA eluted off the CE capillary (˜7.5 minutes) the active DNA was collected by using a pressure rinse with water. FIG. 19 shows an electropherogram of the first CE-SELEX round. CE conditions were PBS buffer with 1 mM MgCl₂, pH=8.0, 30 kV, 25° C., UV detection at 254 nm. The active DNA in the CE fraction was PCR amplified and them made single stranded using the procedures discussed in the experimental section. Prior to performing the second SELEX round, a simple binding assay was done on the ssDNA from the end of the first round to test how well the selection was proceeding. Increasing amounts of IgE were added to 1 μL aliquots of the ssDNA solution. FIG. 20 shows the resulting electropherograms for these samples. CE conditions were PBS buffer with 1 mM MgCl₂, pH=8.0, 30 kV, 25° C., and UV detection at 254 nm. The results from FIG. 20 indicate that 33% of the ssDNA pool binds IgE after only a single round of selection.

For the second CE-SELEX round, 10 μL of the ssDNA from the end of the first round and 0.46 μM of IgE were used. The binding assay using ssDNA from the end of the second round is shown in FIG. 21. CE conditions were PBS buffer with 1 mM MgCl₂, pH=8.0, 30 kV, 25° C., and UV detection at 254 nm. After the second CE-SELEX round, almost 100% of the DNA binds to IgE. This is a very significant result because in conventional SELEX experiments even after 10 or more rounds of selection only ˜50% of the DNA pool shows affinity for the target molecule. The remaining DNA comes through each round as a result of non-specific interactions in the affinity chromatography column used in conventional SELEX.

The ssDNA from the end of the second round of selection was cloned and sequenced. The sequences for the clones obtained from the first CE-SELEX experiment are shown in FIG. 28. A sufficient amount of Clone 1.8 for further study was commercially synthesized by Integrated DNA Technologies Inc., Coralville, Iowa. A fluorescent tag (6-carboxyfluorescein (6-FAM)) was added at the 5′ end of the clone (5′-/6-FAM/AGC AGC ACA GAG GTC AGA TGG TAA TTT TCG CTG CGA TTA GGC TTG TGA CAA AAT TAC TAT CCT ATG CGT GCT ACC GTG AA-3′ (SEQ ID NO:8)) to facilitate detection of the very low concentrations of this clone necessary to measure <mM dissociation constants using ACE. FIG. 22 shows a binding curve for Clone 1.8. A nonlinear least-squares analysis of the binding data in FIG. 22 yielded a K_{d} of 293±248 nM for Clone 1.8.

Second CE-SELEX selection against IgE: For the second CE-SELEX selection with IgE, the first round was done with a sample solution containing 2.25 mM of the 80-base DNA library dissolved in the CE run buffer and 0.34 μM IgE. CE fractions were collected similar to the first CE-SELEX selection with IgE, except that 5 pounds per square inch (psi) of pressure was applied for 18 seconds before collecting the fraction of active DNA. This was done in order to remove any inactive DNA that may have still been on the capillary. From other experiments with CE fraction collections it was observed that the active DNA did not elute off the capillary with an 18 second pressure of 5 psi. Thus, no active DNA sequences were lost in this additional step, but rather, more of the inactive DNA was eliminated. The active DNA in the CE fraction was PCR amplified and made single stranded. FIG. 23 shows electropherograms of the binding assay for the ssDNA from this round of selection. CE conditions were PBS buffer with 1 mM MgCl₂, pH=8.0, 30 kV, 25° C., and UV detection at 254 nm. In this case, 67% of the ssDNA shows an affinity to IgE. This is a higher rate compared to the first round in the first CE-SELEX experiment discussed above. This indicates that a faster rate of enhancement was achieved in the second experiment.

The second round of selection was performed with 10 μL of the ssDNA from the end of the first round plus 0.1 μM IgE. FIG. 24 shows the results of the binding assay with the ssDNA from the end of the second round of selection. CE conditions were PBS buffer with 1 mM MgCl₂, pH=8.0, 30 kV, 25° C., and UV detection at 254 nm. The results of FIG. 24 indicate that after the second round almost 100% of the ssDNA binds to IgE. Approximately 30 clones were synthesized from the ssDNA from the end of the second round. FIG. 29 shows the sequences of the clones from the second experiment. FIG. 25 shows a binding curve used for the calculation of the K_{d} value for one of these clones, Clone 2.27 (5′-/6-FAM/AGC AGC ACA GAG GTC AGA TGG ATG GGG GGG TCT AAC GTG CGA TCT GCC GAC TTT ATC CTG CCT ATG CGT GCT ACC GTG AA-3′(SEQ ID NO:58)). Using the data in FIG. 25, a nonlinear least-squares analysis yielded a K_{d} of 52±51 nM for Clone 2.27. This shows that Clone 2.27 binds IgE more strongly that Clone 1.8. The reason the clone from the second CE-SELEX experiment has a better K_{d} values is most likely a result of the smaller concentrations of IgE used in the second experiment. In the CE-SELEX approach of the present invention, the number of target molecules is much smaller than the number of DNA molecules. The DNA molecules therefore must compete for binding sites on the target. Decreasing the target concentration further intensifies competition for the target molecules even more. It is expected that DNA sequences that bind the target best will be most successful in this competition resulting in selection of better binders as the target concentration is decreased.

As stated earlier, there have been reports of conventional SELEX selections with IgE. So far, the best aptamer for IgE is one with a reported K_{d} of 10 nM. This conventional SELEX aptamer was synthesized by us with a 6-FAM fluorescent tag attached to it (5′-/6-FAM/CTA CCT ACG ATC TGA CTA GCG GGG CAC GTT TAT CCG TCC CTC CTA GTG GCG TGC CCC GCT TAC TCT CAT GTA GTT CC-3′ (SEQ ID NO:127)). The binding curve for the conventional SELEX aptamer is shown in FIG. 26 and a K_{d} value of 4±1 nM was obtained from this curve. This is somewhat similar to the literature K_{d} value for the conventional SELEX aptamer. In the present work the best aptamer obtained has a K_{d} of 52 nM. Thus, while an improved aptamer compared to conventional SELEX was not obtained, almost 100% enrichment of sequences that bind to IgE was achieved, whereas conventional SELEX can only achieve 50% enrichment. Also, only 2 rounds of selection were performed, compared to 15 rounds that were done in the conventional SELEX experiment.

The results for the K_{d} values of the clones from the first and second CE-SELEX experiments show that there is a correlation between the K_{d} values of the clones and the concentration of IgE used in the selection. For the first CE-SELEX selection the concentration of IgE used in the final round was 460 nM and the K_{d} of Clone 1.8 was 293 nM. In the second CE-SELEX experiment, 100 nM of IgE was used in the final round and the K_{d} of Clone 2.27 was 52 nM. With this in mind, a third CE-SELEX selection was performed using a very low amount of IgE, 1 pM, in an attempt to obtain aptamers with better binding affinities for IgE than those obtained thus far.

Third CE-SELEX selection against IgE: For the third CE-SELEX experiment, 2.25 mM of the 80-base DNA library dissolved in CE run buffer and 1 pM of IgE was used in the first CE-SELEX round. FIG. 27 shows electropherograms obtained for the binding assay using ssDNA from the end of the first round which indicate that 62% of the DNA binds IgE. CE conditions were PBS buffer with 1 mM MgCl₂, pH=8.0, 20 kV, 25° C., and UV detection at 254 nm. Three more CE-SELEX rounds were performed, each using 1 pM of the target and 10 μL of the ssDNA from each previous round. The amounts of DNA bound to IgE from after rounds two, three, and four were 96%, ˜100%, and ˜100%, respectively. Based on the injection conditions used here and the capillary dimensions, a 1 pM solution of IgE results in ˜10,000 IgE molecules injected onto the capillary. This means that CE-SELEX is able to collect only 10,000 DNA molecules and the PCR reaction is able to proceed well with this low amount of starting DNA. An injection of 2.25 mM of the DNA library corresponds to ˜1×10¹³ different DNA sequences. Thus, CE-SELEX is able to achieve close to 100% enrichment while selecting only 1×10⁻⁹% of the DNA library and eliminating the rest. To the best of our knowledge, conventional SELEX is not able to separate active DNA from inactive DNA at such a high level of discrimination in a given round.

Approximately 30 clones were obtained from the DNA from the end of round two (FIG. 30) and round four (FIG. 31). To determine if there was any improvement in the K_{d} values of the aptamers after four selection rounds, representative clones from round two and round four will be compared. Representative clones from the end of round two and round four are currently being synthesized and K_{d} values for them will be determined. Since only pM amounts of IgE were used to select these aptamers, it is expected that a big improvement in the binding affinities for these clones compared to the previous two CE-SELEX experiments will be observed.

In this Example the viability of CE-SELEX using selections against IgE have been demonstrated. In all cases, close to 100% enrichment of the DNA is achieved in only two rounds. Aptamers with K_{d} values as low as 52 nM have been obtained and lower K_{d} values are expected for the aptamers from the third CE-SELEX experiment. Two CE-SELEX rounds take about two days to complete whereas 15 conventional SELEX rounds are usually done in a month. Thus, we have significantly shortened the experiment time by using CE-SELEX and achieved a much higher rate of enrichment. If the clones from the third CE-SELEX experiment give us K_{d} values in the pM domain, then we will have been able to show that CE-SELEX is superior to conventional SELEX in almost all aspects.

The complete disclosures of all patents, patent applications including provisional patent applications, and publications, and electronically available material (e.g., GenBank amino acid and nucleotide sequence submissions) cited herein are incorporated by reference. The foregoing detailed description and examples have been provided for clarity of understanding only. No unnecessary limitations are to be understood therefrom. The invention is not limited to the exact details shown and described; many variations will be apparent to one skilled in the art and are intended to be included within the invention defined by the claims.

### Sequence Listing Free Text

SEQ ID NO: 1-117 are aptamer sequences.

SEQ ID NO:118 is a 20-base ssDNA target sequence.

SEQ ID NO:119 is a 70-base ssDNA template sequence.

SEQ ID NO:120 is a 70-base ssDNA control sequence.

SEQ ID NO: 121-124 are primer sequences.

SEQ ID NO:125 is a template DNA sequence.

SEQ ID NO:126 is a DNA library sequence.

SEQ ID NO:127 is a conventional IgE aptamer sequence.

What is claimed is:

1. A method for identifying nucleic acid ligands of a target molecule from a candidate mixture comprised of single stranded nucleic acids each having a region of randomized sequence, said method comprising:
contacting the candidate mixture of single stranded nucleic acids each having a region of randomized sequence with the target molecule, wherein nucleic acids having an increased affinity to the target molecule relative to the candidate mixture may be partitioned from the remainder of the candidate mixture;
partitioning the increased affinity nucleic acids from the remainder of the candidate mixture by capillary electrophoresis; and
amplifying the increased affinity nucleic acids, in vitro, to yield a ligand-enriched mixture of nucleic acids, whereby nucleic acid ligands of the target molecule may be identified.

2. The method of claim 1, wherein said target molecule is a large molecule.

3. The method of claim 2, wherein said large molecule target is selected from the group consisting of IgE, Lrp, E. coli metJ protein, elastase, human immunodeficiency virus reverse transcriptase (HIV-RT), human cytomegalovirus (HCMV), and thrombin.

4. The method of claim 1, wherein said target molecule is a small molecule.

5. The method of claim 4, wherein said small molecule target is selected from the group consisting of ATP, L-arginine, kanamycin, lividomycin, neomycin, nicotinamide (NAD), N-methylmesoporphyrin (NMM), theophylline, tobramycin, D-tryptophan, L-valine, vitamin B12, D-serine, L-serine, and γ-aminobutyric acid (γ-ABA).

6. The method of claim 1, wherein said target molecule is a neurotransmitter or a neuropeptide.

7. The method of claim 1 wherein said target molecule is selected from the group consisting of a virus, a bacterium, a eukaryotic cell, an organelle, and a nanoparticle.

8. The method of claim 1, further comprising repeating the steps of contacting the candidate mixture of single stranded nucleic acids each having a region of randomized sequence with the target molecule, partitioning the increased affinity nucleic acids from the remainder of the candidate mixture by capillary electrophoresis, and amplifying the increased affinity nucleic acids.

9. The method of claim 8, wherein steps of contacting the candidate mixture of single stranded nucleic acids each having a region of randomized sequence with the target molecule, partitioning the increased affinity nucleic acids from the remainder of the candidate mixture by capillary electrophoresis, and amplifying the increased affinity nucleic acids are repeated 2-20 times.

10. The method of claim 1, wherein said single-stranded nucleic acids are deoxyribonucleic acids.

11. The method of claim 1, wherein said single-stranded nucleic acids are modified deoxyribonucleic acids.

12. The method of claim 1, wherein said single-stranded nucleic acids are ribonucleic acids.

13. The method of claim 1, wherein said single-stranded nucleic acids are modified ribonucleic acids.

14. The method of claim 1, wherein the amplifying the increased affinity nucleic acids is by polymerase chain reaction (PCR).

15. The method of claim 14 wherein the polymerase chain reaction is performed with primers with a melting temperature of greater that 52° C. and the PCR annealing reaction is carried out at a temperature of 52° C. or greater.

16. The method of claim 15 wherein the primers have a melting temperature of about 59° C. and the PCR annealing reaction is carried out at a temperature of about 52° C. to about 54° C.

17. The method of claim 1, wherein the partitioning the increased affinity nucleic acids from the remainder of the candidate mixture by capillary electrophoresis is performed in a microfluidic device or chip.

18. The method of claim 1, wherein the partitioning the increased affinity nucleic acids from the remainder of the candidate mixture by capillary electrophoresis is carried out in a CE buffer of about 5 mM to about 40 mM NaCl.

19. The method of claim 18 wherein the CE buffer is about 30 mM NaCl.

20. A method for identifying nucleic acid ligands of a target molecule from a candidate mixture comprised of single stranded nucleic acids each having a region of randomized sequence, said method comprising:
contacting the candidate mixture of single stranded nucleic acids each having a region of randomized sequence with the target molecule, wherein nucleic acids having an increased affinity to the target molecule relative to the candidate mixture may be partitioned from the remainder of the candidate mixture;
partitioning the increased affinity nucleic acids from the remainder of the candidate mixture by capillary electrophoresis;
amplifying the increased affinity nucleic acids, in vitro, to yield a ligand-enriched mixture of nucleic acids; and
identifying a nucleic acid ligand of the target molecule from the ligand-enriched mixture of nucleic acids.

21. A nucleic acid ligand isolated by the method of claim 1.

22. The nucleic acid ligand of claim 21, wherein the nucleic acid ligand is selected from the group consisting of a DNA oligonucleotide, an RNA oligonucleotide, or a modification thereof.

23. The nucleic acid ligand of claim 21, wherein the nucleic acid ligand binds to a large molecule target molecule.

24. The nucleic acid ligand of claim 21, wherein the nucleic acid ligand binds to a small molecule target molecule.

25. The nucleic acid ligand of claim 21, wherein the nucleic acid ligand binds to a macromolecular target.

26. The nucleic acid ligand of claim 21, wherein the nucleic acid ligand has a binding affinity for a target molecule selected from the group consisting of IgE, Lrp, E. coli metJ protein, elastase, human immunodeficiency virus reverse transcriptase (HIV-RT), human cytomegalovirus (HCMV), thrombin, ATP, L-arginine, kanamycin, lividomycin, neomycin, nicotinamide (NAD), N-methylmesoporphyrin (NMM), theophylline, tobramycin, D-tryptophan, L-valine, vitamin B12, D-serine, L-serine, γ-aminobutyric acid (γ-ABA), a virus, a bacteria, a eukaryotic cell, an organelle, and a nanoparticle.

27. An nucleic acid ligand selected from the group consisting of SEQ ID NO:1-117, or a modification thereof.

28. A CE-SELEX kit comprising one or more components selected from the group consisting of capillary tubes suitable for CE, a primer pair, a DNA combinatorial library, an RNA combinatorial library, PCR reagents, CE separation buffer, streptavidin/agarose columns, transcriptase, and reverse transcriptase.

29. The CE-SELEX kit of claim 28 further comprising instructions for use.

30. The CE-SELEX kit of claim 29 wherein the instructions for use comprise instructions for the modification of the instrument control and data collection software of a commercial CE instrument to facilitate fraction collection in the CE-SELEX procedure.


SEQUENCE LISTING (127 sequences)

SEQ ID NO: 1 - DNA, 80 bp, ARTIFICIAL SEQUENCE
  aptamer
agcagcacagaggtcagatggtgacatgcgggctctgggggtacgatgtaagctatgtcccctatgcgtgctaccgtgaa

SEQ ID NO: 2 - DNA, 80 bp, ARTIFICIAL SEQUENCE
  aptamer
agcagcacagaggtcagatgctagcgctattcgtcaccggtttaatatatctcatggatccctatgcgtgctaccgtgaa

SEQ ID NO: 3 - DNA, 80 bp, ARTIFICIAL SEQUENCE
  aptamer
agcagcacagaggtcagatgtgtcgtgacttaagcttccgctgggattgacatctgtggccctatgcgtgctaccgtgaa

SEQ ID NO: 4 - DNA, 80 bp, ARTIFICIAL SEQUENCE
  aptamer
agcagcacagaggtcagatggctttcgacgaacataagcggcaattggggaacattcttacctatgcgtgctaccgtgaa

SEQ ID NO: 5 - DNA, 80 bp, ARTIFICIAL SEQUENCE
  aptamer
agcagcacagaggtcagatgggttggggaagtgtaaacgcagtccgaggttcagaaacaacctatgcgtgctaccgtgaa

SEQ ID NO: 6 - DNA, 80 bp, ARTIFICIAL SEQUENCE
  aptamer
agcagcacagaggtcagatgttgtgtaccgttatttgtgcctcagcatccccgtggctaacctatgcgtgctaccgtgaa

SEQ ID NO: 7 - DNA, 80 bp, ARTIFICIAL SEQUENCE
  aptamer
agcagcacagaggtcagatgggctcgcgttcgctttacaagtatagtctcagaatggactcctatgcgtgctaccgtgaa

SEQ ID NO: 8 - DNA, 80 bp, ARTIFICIAL SEQUENCE
  aptamer
agcagcacagaggtcagatggtaattttcgctgcgattaggcttgtgacaaaattactatcctatgcgtgctaccgtgaa

SEQ ID NO: 9 - DNA, 80 bp, ARTIFICIAL SEQUENCE
  aptamer
agcagcacagaggtcagatgcactcagacgtgtccctcttaggagtattagtaatcgattcctatgcgtgctaccgtgaa

SEQ ID NO: 10 - DNA, 80 bp, ARTIFICIAL SEQUENCE
  aptamer
agcagcacagcggtcagatgtttactcgtgaccgaggtgtcacgtaactaatcagttgtgcctatgcgtgctaccgtgaa

SEQ ID NO: 11 - DNA, 80 bp, ARTIFICIAL SEQUENCE
  aptamer
agcagcacagaggtcagatgataggccatgatccatcgcaacccggatagctgttagtttcctatgcgtgctaccgtgaa

SEQ ID NO: 12 - DNA, 80 bp, ARTIFICIAL SEQUENCE
  aptamer
agcagcacagaggtcagatgtgacatgaggaagggtcgtcagcacggccgctgcaagcgacctatgcgtgctaccgtgaa

SEQ ID NO: 13 - DNA, 80 bp, ARTIFICIAL SEQUENCE
  aptamer
agcagcacagaggtcagatgcataattcttcgaactctgatacggtggattatggcggtacctatgcgtgctaccgtgaa

SEQ ID NO: 14 - DNA, 80 bp, ARTIFICIAL SEQUENCE
  aptamer
agcagcacagaggtcagatgcacacatatgttacacagaagaacggatttaatagcccgccctatgcgtgctaccgtgaa

SEQ ID NO: 15 - DNA, 80 bp, ARTIFICIAL SEQUENCE
  aptamer
agcagcacagaggtcagatggaggaggtcgtgcgcattccaaactagatcatcgaaggtacctatgcgtgctaccgtgaa

SEQ ID NO: 16 - DNA, 80 bp, ARTIFICIAL SEQUENCE
  aptamer
agcagcacagaggtcagatgttctttttggatacgtcagcctccggctgtaaccgtgggtcctatgcgtgctaccgtgaa

SEQ ID NO: 17 - DNA, 80 bp, ARTIFICIAL SEQUENCE
  aptamer
agcagcacagaggtcagatgcagcatctcgggatcgttcgcaagtttctggttgtgttaacctatgcgtgctaccgtgaa

SEQ ID NO: 18 - DNA, 80 bp, ARTIFICIAL SEQUENCE
  aptamer
agcagcacagaggtcagatgacactctggtagtgaacacagcatagtgcacctcatagggcctatgcgtgctaccgtgaa

SEQ ID NO: 19 - DNA, 80 bp, ARTIFICIAL SEQUENCE
  aptamer
agcagcacagaggtcagatgttcgcgagtgtacagttttaactacagatgtggtcgccagcctatgcgtgctaccgtgaa

SEQ ID NO: 20 - DNA, 80 bp, ARTIFICIAL SEQUENCE
  aptamer
agcagcacagaggtcagatgagcgtggtttagctccactttatggatagggacaggggcacctatgcgtgctaccgtgaa

SEQ ID NO: 21 - DNA, 80 bp, ARTIFICIAL SEQUENCE
  aptamer
agcagcacagaggtcagatgctcagaaatttatttcacgtttacgaatcctttcggtagtcctatgcgtgctaccgtgaa

SEQ ID NO: 22 - DNA, 80 bp, ARTIFICIAL SEQUENCE
  aptamer
agcagcacagaggtcagatgttgtgctacaactatgtttatgcgtccgtacgggatcatacctatgcgtgctaccgtgaa

SEQ ID NO: 23 - DNA, 79 bp, ARTIFICIAL SEQUENCE
  aptamer
agcagcacagaggtcagatgagtttttgcccgtagaaccagaagaatgtctcggatcgacctatgcgtgctaccgtgaa

SEQ ID NO: 24 - DNA, 80 bp, ARTIFICIAL SEQUENCE
  aptamer
agcagcacagaggtcagatgctaagaagtacgcattttcaacgtaatccgtaaaaattatcctatgcgtgctaccgtgaa

SEQ ID NO: 25 - DNA, 80 bp, ARTIFICIAL SEQUENCE
  aptamer
agcagcacagaggtcagatgattcctcgaaagaattcggtacgcgccctcttaagggcaacctatgcgtgctaccgtgaa

SEQ ID NO: 26 - DNA, 79 bp, ARTIFICIAL SEQUENCE
  aptamer
agcagcacagaggtcagatgttgtcctacttagtggtcgggccaggaattgtgataacacctatgcgtgctaccgtgaa

SEQ ID NO: 27 - DNA, 80 bp, ARTIFICIAL SEQUENCE
  aptamer
agcagcacagaggtcagatgagataagaaggacagacgtaaggtatgattttgatttggacctatgcgtgctaccgtgaa

SEQ ID NO: 28 - DNA, 80 bp, ARTIFICIAL SEQUENCE
  aptamer
agcagcacagaggtcagatgggccatctttcatgttttagacgttttaaaacgtgggatacctatgcgtgctaccgtgaa

SEQ ID NO: 29 - DNA, 80 bp, ARTIFICIAL SEQUENCE
  aptamer
agcagcacagaggtcagatgaatcaagatcaatggcccatcaatacccagcaggcagactcctatgcgtgctaccgtgaa

SEQ ID NO: 30 - DNA, 70 bp, ARTIFICIAL SEQUENCE
  aptamer
agcagcacagaggtcagatgacccttgagatttagccaaggcatatgattcctatgcgtgctaccgtgaa

SEQ ID NO: 31 - DNA, 80 bp, ARTIFICIAL SEQUENCE
  aptamer
agcagcacagaggtcagatgccacggtcagcttcatacgtggggacttcttgattaggaacctatgcgtgctaccgtgaa

SEQ ID NO: 32 - DNA, 80 bp, ARTIFICIAL SEQUENCE
  aptamer
agcagcacagatgtcagatgatggtagtatataaatgtacatcgaccctgagcgcgaatacctatgcgtgctaccgtgaa

SEQ ID NO: 33 - DNA, 80 bp, ARTIFICIAL SEQUENCE
  aptamer
agcagcacagaggtcagatgaatgtacggatgtagtacacaacggcttgctatctccttccctatgcgtgctgccgtgaa

SEQ ID NO: 34 - DNA, 80 bp, ARTIFICIAL SEQUENCE
  aptamer
agcagcacagaggtcagatgaaacgacgcattaccgggctaaccgggcgcgggagcgcgccctatgcgtgctaccgtgaa

SEQ ID NO: 35 - DNA, 80 bp, ARTIFICIAL SEQUENCE
  aptamer
agcagcacagaggtcagatggtatttataccgcgttatcactgggtctgggtgtaagcttcctatgcgtgctaccgtgaa

SEQ ID NO: 36 - DNA, 79 bp, ARTIFICIAL SEQUENCE
  aptamer
agcagcacagaggtcagatgatcagcgtcgttgggtgaggcgtaagatcttgctaggtacctatgcgtgctaccgtgaa

SEQ ID NO: 37 - DNA, 80 bp, ARTIFICIAL SEQUENCE
  aptamer
agcagcacagaggtcagatgcgttcaattttatgtggggcctaatcaagcgatcttgggacctatgcgtgctaccgtgaa

SEQ ID NO: 38 - DNA, 79 bp, ARTIFICIAL SEQUENCE
  aptamer
agcagcacagaggtcagatgggtccatcgatagcaagtggtattattaatatctgctagcctatgcgtgctaccgtgaa

SEQ ID NO: 39 - DNA, 80 bp, ARTIFICIAL SEQUENCE
  aptamer
agcagcacagaggtcagatgtccaaatcatccatataattggaatagtagcagtatgacccctatgcgtgctaccgtgaa

SEQ ID NO: 40 - DNA, 80 bp, ARTIFICIAL SEQUENCE
  aptamer
agcagcacagaggtcagatggtatacgaggacgcttgcgaggtttacccccgcctgaatgcctatgcgtgctaccgtgaa

SEQ ID NO: 41 - DNA, 80 bp, ARTIFICIAL SEQUENCE
  aptamer
agcagcacagaggtcagatgcaggctgcttaagttcgatcttagcgtacggggcaattcccctatgcgtgctaccgtgaa

SEQ ID NO: 42 - DNA, 80 bp, ARTIFICIAL SEQUENCE
  aptamer
agcagcacagaggtcagatgagctcatagcgctcgtatcttccacatacgataaatgcgtcctatgcgtgctaccgtgaa

SEQ ID NO: 43 - DNA, 80 bp, ARTIFICIAL SEQUENCE
  aptamer
agcagcacagaggtcagatgcacgacgaatctgataccgctctttaacctcagtggaagccctatgcgtgctaccgtgaa

SEQ ID NO: 44 - DNA, 80 bp, ARTIFICIAL SEQUENCE
  aptamer
agcagcacagaggtcagatgctatcactagcacgtggtttctgacagtgttggctcctgacctatgcgtgctaccgtgaa

SEQ ID NO: 45 - DNA, 80 bp, ARTIFICIAL SEQUENCE
  aptamer
agcagcacagaggtcagatggtttgggtcaaatacctcgcctacttgtccacgtgtaaatcctatgcgtgctaccgtgaa

SEQ ID NO: 46 - DNA, 80 bp, ARTIFICIAL SEQUENCE
  aptamer
agcagcacagaggtcagatgtcaccgcattgctcatgggcaccgaacatatggatactcgcctatgcgtgctaccgtgaa

SEQ ID NO: 47 - DNA, 80 bp, ARTIFICIAL SEQUENCE
  aptamer
agcagcacagaggtcagatgacggtatcgatccaacctcgaagcatacaatgggccctcccctatgcgtgctaccgtgaa

SEQ ID NO: 48 - DNA, 80 bp, ARTIFICIAL SEQUENCE
  aptamer
agcagcacagaggtcagatggtggctagttgttcgaaatgcagtccagagatgactacggcctatgcgtgctaccgtgaa

SEQ ID NO: 49 - DNA, 80 bp, ARTIFICIAL SEQUENCE
  aptamer
agcagcacagaggtcagatgggttatacctaggtggcgcatatctccaagtatggacttccctatgcgtgctaccgtgaa

SEQ ID NO: 50 - DNA, 80 bp, ARTIFICIAL SEQUENCE
  aptamer
agcagcacagaggtcagatgctaatccttctgcatgtcagcgtagtataagagggcaggccctatgcgtgctaccgtgaa

SEQ ID NO: 51 - DNA, 80 bp, ARTIFICIAL SEQUENCE
  aptamer
agcagcacagaggtcagatgaaaggtgatgtgtgtttttccaacctgttgtaatatttcacctatgcgtgctaccgtgaa

SEQ ID NO: 52 - DNA, 80 bp, ARTIFICIAL SEQUENCE
  aptamer
agcagcacagaggtcagatggtcgcgttgatcgcttgtttgtataatgcctagttaactgcctatgcgtgctaccgtgaa

SEQ ID NO: 53 - DNA, 78 bp, ARTIFICIAL SEQUENCE
  aptamer
agcagcacagaggtcagatgaaccatgaccctggtgcgactagagcccatcgaacacacctatgcgtgctaccgtgaa

SEQ ID NO: 54 - DNA, 80 bp, ARTIFICIAL SEQUENCE
  aptamer
agcagcacagaggtcagatgacgaccttacaacgttatcctattggcaattcctatttaacctatgcgtgctaccgtgaa

SEQ ID NO: 55 - DNA, 80 bp, ARTIFICIAL SEQUENCE
  aptamer
agcagcacagaggtcagatggtccggagagatctatagaagattatatacatgttaggatcctatgcgtgctaccgtgaa

SEQ ID NO: 56 - DNA, 75 bp, ARTIFICIAL SEQUENCE
  aptamer
agcagcacagaggtcagatggttgaagactccaaatatgatatcgaacgtatatccctatgcgtgctaccgtgaa

SEQ ID NO: 57 - DNA, 80 bp, ARTIFICIAL SEQUENCE
  aptamer
agcagcacagaggtcagatgcgtagatcctgagcaaggctttacgtcgatatgtccgggtcctatgcgtgctaccgtgaa

SEQ ID NO: 58 - DNA, 80 bp, ARTIFICIAL SEQUENCE
  aptamer
agcagcacagaggtcagatggatgggggggtctaacgtgcgatctgccgactttatcctgcctatgcgtgctaccgtgaa

SEQ ID NO: 59 - DNA, 80 bp, ARTIFICIAL SEQUENCE
  aptamer
agcagcacagaggtcagatggcgcctgtcatcacttttgtttcttccgcaattcctttgtcctatgcgtgctaccgtgaa

SEQ ID NO: 60 - DNA, 80 bp, ARTIFICIAL SEQUENCE
  aptamer
agcagcacagaggtcagatgaaaattgatgatggtacctatctcgggtgaccaaacagttcctatgcgtgctaccgtgaa

SEQ ID NO: 61 - DNA, 80 bp, ARTIFICIAL SEQUENCE
  aptamer
agcagcacagaggtcagatgactttttccgtttatttctgcgtatatcttgccagcatttcctatgcgtgctaccgtgaa

SEQ ID NO: 62 - DNA, 80 bp, ARTIFICIAL SEQUENCE
  aptamer
agcagcacagaggtcagatggcgtatcgaaaagtcattaaagggctcattttatatcgtacctatgcgtgctaccgtgaa

SEQ ID NO: 63 - DNA, 80 bp, ARTIFICIAL SEQUENCE
  aptamer
agcagcacagaggtcagatgtacctggtttcggcttaattgtcgcgatatcaatttaagacctatgcgtgctaccgtgaa

SEQ ID NO: 64 - DNA, 80 bp, ARTIFICIAL SEQUENCE
  aptamer
agcagcacagaggtcagatgtaaactcgcatggtggcttcaataataattgtcacaagggcctatgcgtgctaccgtgaa

SEQ ID NO: 65 - DNA, 80 bp, ARTIFICIAL SEQUENCE
  aptamer
agcagcacagaggtcagatgtgttcggtatgctgagctcgtctaatatggtttcatgttccctatgcgtgctaccgtgaa

SEQ ID NO: 66 - DNA, 80 bp, ARTIFICIAL SEQUENCE
  aptamer
agcagcacagaggtcagatgtggaagcagcctacattaacctgcagtccctaagcgtcggcctatgcgtgctaccgtgaa

SEQ ID NO: 67 - DNA, 78 bp, ARTIFICIAL SEQUENCE
  aptamer
agcagcacagaggtcagatgcgagggtgcccctatattttttaagcccgccgggatgtcctatgcgtgctaccgtgaa

SEQ ID NO: 68 - DNA, 80 bp, ARTIFICIAL SEQUENCE
  aptamer
agcagcacagaggtcagatgcgaccagagagtcggtaacgatgcaaatactagggctgatcctatgcgtgctaccgtgaa

SEQ ID NO: 69 - DNA, 80 bp, ARTIFICIAL SEQUENCE
  aptamer
agcagcacagaggtcagatggccatttgcacccttagaacttgtcgactagattatcgtgcctatgcgtgctaccgtgaa

SEQ ID NO: 70 - DNA, 80 bp, ARTIFICIAL SEQUENCE
  aptamer
agcagcacagaggtcagatgtgcagatcggtgatacggggaatgtacgggcgctaattgacctatgcgtgctaccgtgaa

SEQ ID NO: 71 - DNA, 80 bp, ARTIFICIAL SEQUENCE
  aptamer
agcagcacagaggtcagatggtgcggggtaggtggcgtttgatatttgagccttttaatacctatgcgtgctaccgtgaa

SEQ ID NO: 72 - DNA, 80 bp, ARTIFICIAL SEQUENCE
  aptamer
agcagcacagaggtcagatgcaaggatacaagtataggtcgcagttcgacgtttacagcacctatgcgtgctaccgtgaa

SEQ ID NO: 73 - DNA, 80 bp, ARTIFICIAL SEQUENCE
  aptamer
agcagcacagaggtcagatggttactgcggggtatggggactggttgcgtggcttggtgtcctatgcgtgctaccgtgaa

SEQ ID NO: 74 - DNA, 78 bp, ARTIFICIAL SEQUENCE
  aptamer
cagcacagaggtcagatgattagatgggggctgatatctgggatgcctgctcacgaatcctatgcgtgctaccgtgaa

SEQ ID NO: 75 - DNA, 80 bp, ARTIFICIAL SEQUENCE
  aptamer
agcagcacagaggtcagatgcgaaaagtgcggtaaggattacatttacggtggagatttccctatgcgtgctaccgtgaa

SEQ ID NO: 76 - DNA, 80 bp, ARTIFICIAL SEQUENCE
  aptamer
agcagcacagaggtcagatgcctttgcttctggtaggttcaacactatcgcatactacatcctatgcgtgctaccgtgaa

SEQ ID NO: 77 - DNA, 80 bp, ARTIFICIAL SEQUENCE
  aptamer
agcagcacagaggtcagatgaagcgattgtaatcaacgccgtaagacctcgagtcgatttcctatgcgtgctaccgtgaa

SEQ ID NO: 78 - DNA, 80 bp, ARTIFICIAL SEQUENCE
  aptamer
agcagcacagaggtcagatgatcgatacagtggtgttagatggaatttcttggtataagacctatgcgtgctaccgtgaa

SEQ ID NO: 79 - DNA, 80 bp, ARTIFICIAL SEQUENCE
  aptamer
agcagcacagaggtcagatggcagatagcgcgtcgtccaccttcggcgtgaaaaaaccgtcctatgcgtgctaccgtgaa

SEQ ID NO: 80 - DNA, 79 bp, ARTIFICIAL SEQUENCE
  aptamer
agcagcacagaggtcagatgtgaacagcctaccccatattacgtttctaaaattatgaacctatgcgtgctaccgtgaa

SEQ ID NO: 81 - DNA, 80 bp, ARTIFICIAL SEQUENCE
  aptamer
agcagcacagaggtcagatgtcaaaaaaaatcagcacgggataaatgaacgacatgggatcctatgcgtgctaccgtgaa

SEQ ID NO: 82 - DNA, 80 bp, ARTIFICIAL SEQUENCE
  aptamer
agcagcacagaggtcagatgctgaagtcccatcgtctactcgggcttggtcaatatgtcgcctatgcgtgctaccgtgaa

SEQ ID NO: 83 - DNA, 80 bp, ARTIFICIAL SEQUENCE
  aptamer
agcagcacagaggtcagatgaaagtattgacgtgttctgcagtcatgtttacagaagccgcctatgcgtgctaccgtgaa

SEQ ID NO: 84 - DNA, 80 bp, ARTIFICIAL SEQUENCE
  aptamer
agcagcacagaggtcagatgcgcgcagaattttgagtcatgtactaaggaattgattggtcctatgcgtgctaccgtgaa

SEQ ID NO: 85 - DNA, 79 bp, ARTIFICIAL SEQUENCE
  aptamer
agcagcacagaggtcagatgggcccatggctactttttatacgtgcgtcacgcagtatgcctatgcgtgctaccgtgaa

SEQ ID NO: 86 - DNA, 80 bp, ARTIFICIAL SEQUENCE
  aptamer
agcagcacagaggtcagatgttcttgccgccttcataatacatctgcataaggacaagatcctatgcgtgctaccgtgaa

SEQ ID NO: 87 - DNA, 80 bp, ARTIFICIAL SEQUENCE
  aptamer
agcagcacagaggtcagatgttactttacattcttatggacagaaactgtgaatcacactcctatgcgtgctaccgtgaa

SEQ ID NO: 88 - DNA, 80 bp, ARTIFICIAL SEQUENCE
  aptamer
agcagcacagaggtcagatgcgtcgtgtaaacactggattgtttaacggagtactggaaccctatgcgtgctaccgtgaa

SEQ ID NO: 89 - DNA, 80 bp, ARTIFICIAL SEQUENCE
  aptamer
agcagcacagaggtcagatgatcccatgtctgttattgctcaatgggtatacacactcggcctatgcgtgctaccgtgaa

SEQ ID NO: 90 - DNA, 79 bp, ARTIFICIAL SEQUENCE
  aptamer
agcagcacagaggtcagatgattttaatcacgttctgttcagacaggtaatgacctgtccctatgcgtgctaccgtgaa

SEQ ID NO: 91 - DNA, 80 bp, ARTIFICIAL SEQUENCE
  aptamer
agcagcacagaggtcagatggggagtcaccgaacgaaggaatcaaaagctctaatacgtacctatgcgtgctaccgtgaa

SEQ ID NO: 92 - DNA, 80 bp, ARTIFICIAL SEQUENCE
  aptamer
agcagcacagaggtcagatggaaaatccgacaagagagtaccagctcagagcccccgccccctatgcgtgctaccgtgaa

SEQ ID NO: 93 - DNA, 80 bp, ARTIFICIAL SEQUENCE
  aptamer
agcagcacagaggtcagatgaggttgaacttttaggttacgactctcatagtgttctgcgcctatgcgtgctaccgtgaa

SEQ ID NO: 94 - DNA, 80 bp, ARTIFICIAL SEQUENCE
  aptamer
agcagcacagaggtcagatgaatgttacgacgtctaaggaacaatatcttctatctaaggcctatgcgtgctaccgtgaa

SEQ ID NO: 95 - DNA, 80 bp, ARTIFICIAL SEQUENCE
  aptamer
agcagcacagaggtcagatgcaccacattccgcgagatatatgctccttttggtttatcccctatgcgtgctaccgtgaa

SEQ ID NO: 96 - DNA, 80 bp, ARTIFICIAL SEQUENCE
  aptamer
agcagcacagaggtcagatgttaactcgccgttctgccagtcagttgtctctttcgtattcctatgcgtgctaccgtgaa

SEQ ID NO: 97 - DNA, 80 bp, ARTIFICIAL SEQUENCE
  aptamer
agcagcacagaggtcagatgtcagggctcagcaatcaatgcagtcaggacacccacttggcctatgcgtgctaccgtgaa

SEQ ID NO: 98 - DNA, 80 bp, ARTIFICIAL SEQUENCE
  aptamer
agcagcacagaggtcagatggttcgttgtagcgcataaagtttatctctcccatgattcacctatgcgtgctaccgtgaa

SEQ ID NO: 99 - DNA, 80 bp, ARTIFICIAL SEQUENCE
  aptamer
agcagcacagaggtcagatgatgtctgatgcgatattctggtcaaaccattggcaagagacctatgcgtgctaccgtgaa

SEQ ID NO: 100 - DNA, 80 bp, ARTIFICIAL SEQUENCE
  aptamer
agcagcacagaggtcagatgcattctttcgagcaacaggtaacagtctattagccgtgaccctatgcgtgctaccgtgaa

SEQ ID NO: 101 - DNA, 79 bp, ARTIFICIAL SEQUENCE
  aptamer
agcagcacagaggtcagatgtagagctttccgtcatatcagtatagataacatatattccctatgcgtgctaccgtgaa

SEQ ID NO: 102 - DNA, 80 bp, ARTIFICIAL SEQUENCE
  aptamer
agcagcacagaggtcagatgggtaggtagccgtgccggttgtgccattgattgtacagttcctatgcgtgctaccgtgaa

SEQ ID NO: 103 - DNA, 80 bp, ARTIFICIAL SEQUENCE
  aptamer
agcagcacagaggtcagatgcgagatggtgtgtgtggaatgaagtcctcgctgtgttttacctatgcgtgctaccgtgaa

SEQ ID NO: 104 - DNA, 80 bp, ARTIFICIAL SEQUENCE
  aptamer
agcagcacagaggtcagatgcacgaggatttttagtaatatttttttagttgcgtgataacctatgcgtgctaccgtgaa

SEQ ID NO: 105 - DNA, 79 bp, ARTIFICIAL SEQUENCE
  aptamer
agcagcacagaggtcagatggctatcttgcgtgagtacagtgtccctgcattgtcgatacctatgcgtgctaccgtaaa

SEQ ID NO: 106 - DNA, 80 bp, ARTIFICIAL SEQUENCE
  aptamer
agcagcacagaggtcagatgcgcatgaaaattatctctcgagttcttatagccttttggacctatgcgtgctaccgtgaa

SEQ ID NO: 107 - DNA, 80 bp, ARTIFICIAL SEQUENCE
  aptamer
agcagcacagaggtcagatggcctatatttataaagacaggagagtaatgtcgagcagaacctatgcgtgctaccgtgaa

SEQ ID NO: 108 - DNA, 80 bp, ARTIFICIAL SEQUENCE
  aptamer
agcagcacagaggtcagatgtatagagaggaggaagcgttccaactgggtaatttggtaccctatgcgtgctaccgtgaa

SEQ ID NO: 109 - DNA, 80 bp, ARTIFICIAL SEQUENCE
  aptamer
agcagcacagaggtcagatggggtggccccgtggtggctgtacgttatcatctgtcatcgcctatgcgtgctaccgtgaa

SEQ ID NO: 110 - DNA, 80 bp, ARTIFICIAL SEQUENCE
  aptamer
agcagcacagaggtcagatgttacaataatgtactactggaaggttgcttctaactgtgacctatgcgtgctaccgtgaa

SEQ ID NO: 111 - DNA, 80 bp, ARTIFICIAL SEQUENCE
  aptamer
agcagcacagaggtcagatgatgctgagaacctgctaagggattccagataaggaggttgcctatgcgtgctaccgtgaa

SEQ ID NO: 112 - DNA, 80 bp, ARTIFICIAL SEQUENCE
  aptamer
agcagcacagaggtcagatgaagggaaattccattccttaagttagaccatacgtatacacctatgcgtgctaccgtgaa

SEQ ID NO: 113 - DNA, 80 bp, ARTIFICIAL SEQUENCE
  aptamer
agcagcacagaggtcagatgaggtggaatgaggtttcccgggatgtacgctgttgtacggcctatgcgtgctaccgtgaa

SEQ ID NO: 114 - DNA, 80 bp, ARTIFICIAL SEQUENCE
  aptamer
agcagcacagaggtcagatgttaaagttgttgatgtcctctctttggggacttattaacgcctatgcgtgctaccgtgaa

SEQ ID NO: 115 - DNA, 80 bp, ARTIFICIAL SEQUENCE
  aptamer
agcagcacagaggtcagatgttgttttcgagtccaggatcaaccaggttctagtcgttaacctatgcgtgctaccgtgaa

SEQ ID NO: 116 - DNA, 80 bp, ARTIFICIAL SEQUENCE
  aptamer
agcagcacagaggtcagatgggatattaatatacttgatgacacggggctagctctgtgacctatgcgtgctaccgtgaa

SEQ ID NO: 117 - DNA, 80 bp, ARTIFICIAL SEQUENCE
  aptamer
agcagcacagaggtcagatgtactggctcccgagttcagaaccctacttgcgaaaagtaccctatgcgtgctaccgtgaa

SEQ ID NO: 118 - DNA, 20 bp, ARTIFICIAL SEQUENCE
  20-base ssDNA target sequence
catgggccaagcttcttcgg

SEQ ID NO: 119 - DNA, 70 bp, ARTIFICIAL SEQUENCE
  70-base ssDNA template sequence
ctacctacgatctgactagctttttccgaagaagcttggcccatgtttttgcttactctcatgtagttcc

SEQ ID NO: 120 - DNA, 70 bp, ARTIFICIAL SEQUENCE
  70-base ssDNA control sequence
ctacctacgatctgactagcttttttttttttttttttttttttttttttgcttactctcatgtagttcc

SEQ ID NO: 121 - DNA, 20 bp, ARTIFICIAL SEQUENCE
  primer
ctacctacgatctgactagc

SEQ ID NO: 122 - DNA, 20 bp, ARTIFICIAL SEQUENCE
  primer
ggaactacatgagagtaagc

SEQ ID NO: 123 - DNA, 20 bp, ARTIFICIAL SEQUENCE
  primer
agcagcacagaggtcagatg

SEQ ID NO: 124 - DNA, 20 bp, ARTIFICIAL SEQUENCE
  primer
ttcacggtagcacgcatagg

SEQ ID NO: 125 - DNA, 70 bp, ARTIFICIAL SEQUENCE
  template DNA sequence
agcagcacagaggtcagatgtttttccgaagaagcttggcccatgtttttcctatgcgtgctaccgtgaa

SEQ ID NO: 126 - DNA, 80 bp, ARTIFICIAL SEQUENCE
  DNA library sequence
agcagcacagaggtcagatgnnnnnnnnnnnnnnnnnnnnnnnnnnnnnnnnnnnnnnnncctatgcgtgctaccgtgaa

SEQ ID NO: 127 - DNA, 77 bp, ARTIFICIAL SEQUENCE
  conventional IgE aptamer
ctacctacgatctgactagcggggcacgtttatccgtccctcctagtggcgtgccccgcttactctcatgtagttcc